(19) United States
(12) Patent Application Publication
(10) Pub. No.: US 2014/0194353 A1
(43) Pub. Date: Jul. 10, 2014

(54) COMPOSITIONS AND METHODS FOR THE TREATMENT OF NERVOUS DISORDERS ASSOCIATED WITH DIABETES

(71) Applicants: C. Ronald Kahn, West Newton, MA (US); Ryo Suzuki, Tokyo (JP)

(72) Inventors: C. Ronald Kahn, West Newton, MA (US); Ryo Suzuki, Tokyo (JP)

(73) Assignee: Joslin Diabetes Center, Inc., Boston, MA (US)

(21) Appl. No.: 13/990,523

(22) PCT Filed: Nov. 29, 2011
(86) PCT No.: PCT/US11/62366

§ 371 Date: Feb. 10, 2014

Related U.S. Application Data
(60) Provisional application No. 61/418,400, filed on Nov. 30, 2010.

(51) Int. Cl. A61K 38/28; G01N 33/53

(52) U.S. Cl. CPC A61K 38/28; G01N 33/53
USPC 514/5.9; 435/7.1

(57) ABSTRACT

Compositions and methods for treating neural dysfunction. A exemplary method comprises administering to a subject having a neuropathy, e.g., a cognitive dysfunction or Alzheimer's, a therapeutically effective amount of an insulin or insulin analog, wherein the insulin or insulin analog crosses the BBB and/or a compound that increases SREBP-2 expression or activity in the CNS of the subject.

### CROSS-REFERENCE TO RELATED APPLICATIONS

This application claims priority to U.S. Provisional application No. 61/418,400, filed Nov. 30, 2010, the contents of which are specifically incorporated by reference herein.

### FEDERALLY SPONSORED RESEARCH OR DEVELOPMENT

This invention was made with Government support under Grant No. DK31036, awarded by the National Institutes of Health. The Government has certain rights in the invention.

### TECHNICAL FIELD

This invention relates to compositions and methods for the treatment of nervous complications (e.g., dysfunction, disorders, conditions, and/or diseases) associated with diabetes (e.g., type I and type II diabetes) including but not limited to diabetic neuropathy or neuropathies.

### BACKGROUND

Type I (insulin-dependent) diabetes and type II (insulin-independent) diabetes are associated with numerous medical complications that affect various tissues of the body. These complications extend to the nervous system to include conditions ranging from acute alterations in mental status due to poor metabolic control to greater rates of decline in cognitive function with age, higher prevalence of depression, increased risk of Alzheimer's disease and other forms of neurological dysfunction and diabetic neuropathy and those disorders associated with diabetic neuropathy (see, e.g., Biessels et al., Lancet Neurol., 7:184-190 (2008); Cukierman et al., Diabetologia 48, 2460-2469 (2005); Ali et al., Diabet Med 23, 1165-1173 (2006); Craft and Watson, Lancet Neurol 3, 169-178 (2004)). Compositions and methods for treating these diabetes-associated nervous disorders are required.

### SUMMARY

The present disclosure provides compositions and methods for treating (e.g., selecting and treating) a subject having a nervous complication, e.g., associated with diabetes.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic showing the pathway leading from diabetes to nervous complications.

FIG. 2 is a schematic showing the cholesterol synthesis pathway.

FIG. 3 is a heat map showing the suppression of cholesterol synthesis pathway in the hypothalamus of STZ-diabetic mice based on microarray data. Each column represents one Affymetrix chip hybridized using the cRNA synthesized from one mouse hypothalamus. Blood glucose levels at the time of sacrifice are indicated above the columns. “Cholesterol biosynthesis” gene sets were significantly reduced in the STZ group compared to the others (nominal P-value<0.001, FDR Q-value=0.033, FWER P-value=0.023, as analyzed by GSEA v2.0 software). Colors represent gene expression values in individual subject expression changes relative to the mean, with red representing higher and blue representing lower expression.

FIG. 4 is a bar graph showing comparison of gene expression for the cholesterol synthetic enzymes in hypothalami of control (CON, n=6), STZ-diabetic (STZ, n=5), and insulin-treated STZ (STZ+INS, n=7) mice. Hypothalami were collected from male C57Bl/6 mice 9 days after STZ injection. Expression was measure by qPCR, and average values in CON assigned a value of 1. Bars denote mean±SEM. a, P<0.01 between CON and STZ; b, P<0.01 between STZ and STZ+INS by ANOVA.

FIG. 5 is a bar graph showing expression levels of SREBP isoforms in the hypothalamus normalized for Tbp.

FIG. 6 is an image of an immunoblot of nuclear extracts from hypothalamic of control and STZ-diabetic mice for nuclear SREBP (nSREBP) indicated by arrows. Lamenin was used as a loading control. *, P<0.05; **, P<0.01; ***, P<0.001 by t-test.

FIG. 7A is a bar graph showing densitometry analysis of the immunoblot shown in FIG. 7B. Cytoplasmic extracts were used and values were normalized using actin as a loading control.

FIG. 7B is an image of an immunoblot.

FIG. 8 is a bar graph showing a comparison of the major cholesterologenic genes and Srebf2 gene expression in 5-month-old prediabetic NOD (n=6) and diabetic NOD (n=5) mouse hypothalami, as assessed by qPCR.

FIG. 9 is a bar graph showing gene expression in hypothalamic of 9-week-old db/+(n=6) and obese diabetic db/db (n=6) mice on a C57Bl/Ks background, as assessed by qPCR.

FIGS. 10A-10B are bar graphs showing comparisons of the major cholesterologenic enzymes and SREBP-2 gene expression in hypothalami from chow-diet-fed (n=6) and high-fat-fed (n=6) (A) C57Bl/6 mice was performed by qPCR. (B) Gene expression in hypothalamus of control (n=6) and ob/ob (n=5) mice was also assessed (B).

FIG. 10C is a bar graph showing the effects of 24 hours of fasting and brain-insulin receptor ablation on gene expression in the hypothalamus (n=7 in each group). Average expression values in fed control hypothalamic were assigned a value of 1.

FIG. 10D is a bar graph showing cholesterol synthetic gene regulation by 24 hours fasting was investigated in the brain-specific insulin receptor knockout (NIRKO) and control mouse hypothalami (n=7 in each group). Average expression values in fed control hypothalami are indicated as 1.0. *, P<0.05; **, P<0.01; ***, P<0.001 between fed and fasted states in each genotype. §, P<0.05 between control and NIRKO hypothalami in a correspondent feeding status. ANOVA was employed to compare the 2 (genotypes)×2 (feeding status) groups.

FIG. 11 is a bar graph showing gene expression in the cerebral cortices of STZ-diabetic mice, as assessed by qPCR.

FIG. 12A is a bar graph showing densitometry analysis of the Western blot shown in FIG. 12B. Values were normalized using actin.

FIG. 12B is an image of a Western blot.

FIG. 13A is a line graph showing blood glucose levels (a, P<0.05 between CON+VEH and STZ+VEH; b, P<0.05 between STZ+VEH and STZ+PHZ by ANOVA).

FIG. 13B is a bar graph showing comparison of gene expression for Srebf2 and its major downstream genes in hypothalami of VEH-treated control (CON+VEH, n=5), VEH-treated STZ-diabetic (STZ+VEH, n=6), and PHZ-treated STZ (STZ+PHZ, n=5) mouse hypothalami was performed by qPCR. In the right panel, blood glucose levels were measured.

FIG. 14 A is a bar graph showing gene expression in hypothalami of vehicle-injected control (CON+VEH icy, n=4), vehicle-injected STZ-diabetic (STZ+VEH icy, n=5), and insulin-injected STZ (STZ+INS icy, n=4) mouse hypothalami was assessed by qPCR.

FIG. 14B is a bar graph showing blood glucose levels were measured 4 hours after the last ICV injection. a, P<0.05 between CON+VEH icy and STZ+VEH icy; b, P<0.05 between STZ+VEH icy and STZ+PHZ icy by ANOVA.

FIGS. 15A-15B are bar graphs showing the effects of the insulin ICV injection on feeding behavior and neuropeptide gene expression. Comparison of gene expression for neuropeptides regulating appetite was performed by qPCR (A). Food intake was measured after the ICV injection (B). Error bars represent SEM.

FIG. 16A is a schematic showing how in vivo cholesterol synthesis in the whole cerebra was assessed for control (n=4) and STZ-diabetic (n=4) mice 17 days after STZ administration using the protocol shown on the left. Cerebra were dissected 1 h after intraperitoneal injection of tritiated water, and cholesterol was isolated by thin layer chromatography (TLC) from the extracted lipid.

FIG. 16B is a bar graph showing the rate of cholesterol synthesis expressed as nmol cholesterol synthesized per gram of cerebrum per hour.

FIG. 16C is a schematic showing how cholesterol content in the synaptosomal membrane extracted from the frontal cortex of control (n=4) and STZ-diabetic (n=4) mice 18 days after STZ administration was assessed. Synaptosome-rich fraction was separated from myelin fraction by discontinuous sucrose gradient centrifugation as shown on the left (Kolomiytseva et al., 2008).

FIG. 16D is a bar graph showing synaptosomal cholesterol content in μg per mg protein.

FIGS. 17A-D are line graphs showing cholesterologenic gene expression and synaptosomal membrane cholesterol in human brain. (A-B) show that cholesterologenic gene expression and synaptosomal membrane cholesterol were positively correlated in these human brain samples. Correlation between Srebf2 and cholesterologenic genes between Srebf2 and Fdps expression (A) and between Srebf2 and Hmgcr expression (B). (C-D) Correlation between Fdps (C) or Hmgcr (D) expression and cholesterol content in the isolated synaptosomal membranes in human cerebral cortices (n=16). Statistical significance in Pearson's correlation coefficient (r) was determined by F-test. Four subgroups are indicated; “Normal”, “DM”, “Dementia”, and “Dementia+DM”. Among the subgroups, no significant difference was observed on gene expression or cholesterol content.

FIG. 18 is a bar graph showing content of representative sterols in the brain measured by HPLC/MS (McDonald et al., 2007) for control (n=4) and STZ-diabetic (n=4) mice 18 days after STZ injection. 24-OH-chol, 24-hydroxycholesterol; 24,25-ep-chol, 24,25-epoxycholesterol; 27-OH-chol, 27-hydroxycholesterol. *, P<0.05; ***, P<0.001 by t-test. Error bars represent SEM.

FIGS. 19A-19B are bar graphs and a corresponding Western blot showing expression of cholesterol 24-hydroxylase (CYP46A1) in STZ-diabetic mouse hypothalami as assessed by qPCR (A) and Western blot (B). *, P<0.05 by t-test.

FIGS. 20A-20B are bar graphs showing that insulin induces cholesterol synthetic gene expression in mouse primary cultured glia and neurons. (A) Primary mouse cultured cortical neurons and glia (18 days in vitro) were incubated in medium with insulin for 6 h. Data are representative of three experiments. (B) Primary culture cortical neurons and glia were incubated with low (5 mM) or high (25 mM) concentrations of glucose in medium for 72 hours. In all panels, mRNA was extracted from the cells at the end of treatment and gene expression levels were quantified by qPCR. *, P<0.05; **, P<0.01; ***, P<0.001 by ANOVA. Error bars represent SEM.

FIGS. 21A-21B are bar graphs showing pharmacological inhibition of insulin signaling pathways in mouse primary cultured glia (see FIGS. 20A-20B). (A) Glia cells were incubated with medium containing DMSO (control), a PI 3-kinase inhibitor LY294002 (LY), a MEK inhibitor U0126 (U), and/or an mTORC1 inhibitor rapamycin (Rapa) prior to insulin stimulation. #, P<0.05 (comparison with DMSO/insulin treated group). *, P<0.05; **, P<0.01 by ANOVA. (B) Rapamycin treatment (0.5 μM) was followed by incubation with a GSK3 inhibitor SB216763 (SB) and/or insulin. *, P<0.05; **, P<0.01 by ANOVA (comparison with DMSO/DMSO treated groups (white bars)).

FIG. 22 is a bar graph showing cholesterol synthetic gene regulation by 24 hours fasting was investigated in the brain-specific insulin receptor knockout (NIRKO) and control mouse hypothalami (n=7 in each group). Average expression values in fed control hypothalami are indicated as 1.0. *, P<0.05; **, P<0.01; ***, P<0.001 between fed and fasted states in each genotype. §, P<0.05 between control and NIRKO hypothalami in a correspondent feeding status. ANOVA was employed to compare the 2 (genotypes)×2 (feeding status) groups.

FIG. 23A is a schematic of the pGIPZ-shSREBP2 construct. pCMV, cytomegalovirus promoter; GFP, green fluorescent protein; IRES, internal ribosome entry site; PuroR, puromycin resistance gene; LTR, long terminal repeat; SIN-LTR, self inactivating LTR.

FIG. 23B is an image of a Western blot of murine hypothalamic neuronal N-25/2 cells with control non-silencing (NS) and shSREBP2 lentivirus infection.

FIG. 23C is a bar graph showing expression levels of Srebf2 and Hmgcr in the primary cultured mouse hippocampal neurons after the lentivirus infection. Values were normalized for Tbp expression.

FIGS. 24A and 24C are images of immunostained cells. (A) Marker staining in primary cultured mouse hippocampal neurons (8 days in vitro) after lentivirus-mediated SREBP-2 silencing. Red represents PSD95, green derives from GFP that the vectors encode, and blue represents the neuron marker MAP2. (C) Staining for the synaptic vesicle marker VAMP2 in the hippocampal neurons. Red represents VAMP2, and the other colors are as above.

FIG. 24 B is a bar graph showing PSD95 density in neurites calculated as signal-positive area divided by the length of neurite. Values were measured in 69 neurites from 30 neurons (Lenti-NS) and 78 neurites from 27 neurons (Lenti-shSREBP2) using ImageJ software. Scale bar, 25 μm.

FIG. 24D is a bar graph showing average VAMP2 staining intensity in the neurites has been calculated using 65 neurites from 35 neurons for the Lenti-NS: control and 49 neurites from 20 neurons for the Lenti-shSREBP2 infected cells. Data are representative of three independent experiments. ***, P<0.001 by t-test. Error bars represent SEM.

FIG. 25A is an image of a Western blot showing SREBP-2 knockdown in the hypothalamus affects feeding behavior and metabolic phenotype.

FIG. 25B is 2 images of immunostained lentivirus-infected hypothalami on day 7. In the left panel, red represents a neuronal marker MAP2, green derives from GFP that the vectors encode, and blue represents nuclei. In the right panel, red represents the astrocyte marker GFAP. Arrows indicate the GFP-positive astrocyte processes. Scale bar, 25 μm.

FIG. 25C is an image showing GFP fluorescence in hypothalami from mice with intrahypothalamic (ihp) lentivirus injection. PVH, paraventricular hypothalamus; VMH, ventromedial hypothalamus; ARC, arcuate nucleus; 3V, the third ventricle.

FIG. 25D is a line graph showing food intake of the male C57Bl/6 mice with ihp injection of Lenti-shSREBP2 (n=18) and control Lenti-NS (n=19). Food was measured twice a day from day 15 after the injection for consecutive 12 days.

FIGS. 26A-26C are graphs showing (A) food intake of male C57Bl/6 mice after ihp injection of Lenti-shSREBP2 (n=18) or control Lenti-NS (n=19). Food intake was measured twice a day from day 15 after the injection for consecutive 12 days. (B and C) Body weight change and absolute body weight of the same mice.

FIG. 27A is a bar graph showing plasma norepinephrine concentrations in mice with ihp injection of Lenti-NS (n=10) and Lenti-shSREBP2 (n=10) after 48 hours of fasting.

FIG. 27B is a bar graph showing plasma glucagon concentrations in mice with ihp injection of Lenti-NS (n=10) and Lenti-shSREBP2 (n=10) after 24 hours of fasting.

FIG. 27C is a bar graph showing fasting plasma insulin concentrations (24 h) in mice with ihp injection of Lenti-NS (n=10) and Lenti-shSREBP2 (n=10). *, P<0.05; **, P<0.01 by t-test. Error bars represent SEM.

FIGS. 27D and 27E are line graphs showing (D) Insulin tolerance tests and (E) glucose tolerance tests of the mice with ihp injection of Lenti-shSREBP2 (n=10) and control Lenti-NS (n=10).

FIGS. 28A-28B are bar graphs showing activity and oxygen consumption levels of the mice with ihp injection of Lenti-shSREBP2 (n=8) and control Lenti-NS (n=8) during 72 hours in metabolic chambers. *, P<0.05 by t-test.

FIG. 29 is a schematic showing a model for cerebral dysfunction in diabetes via insulin-mediated cholesterol regulation. Reduction in circulating insulin results in reduced SREBP-2 in both neurons and astroglial cells, and this results in a reduction of cholesterol synthesis and this causes changes in synapse components, affecting neural excitability and functions.

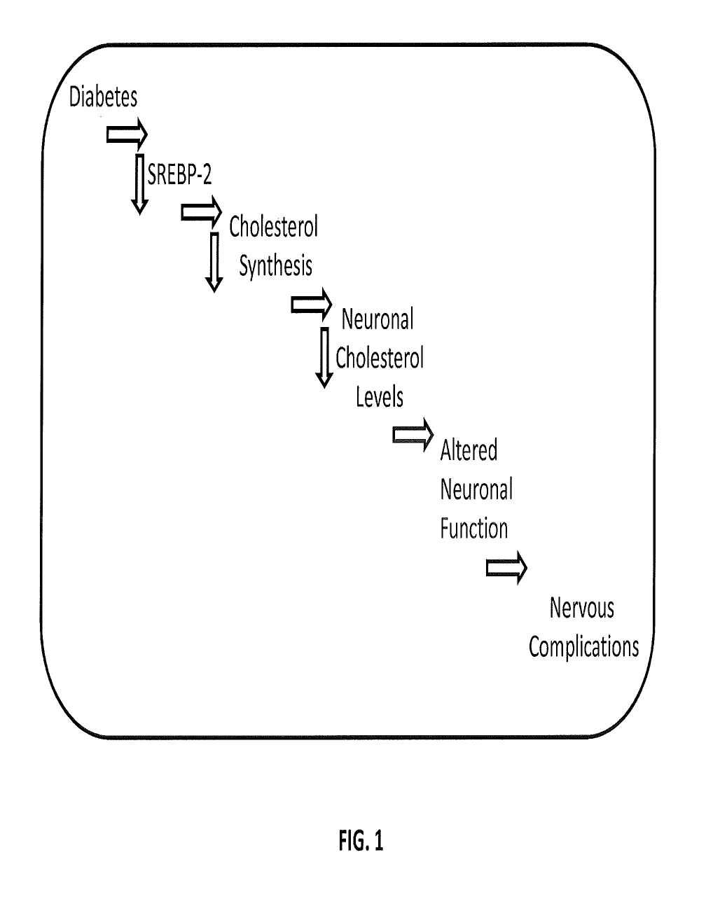

### DETAILED DESCRIPTION

The present disclosure is based, inter alia, on the surprising observation that diabetes (e.g., uncontrolled diabetes) and/or altered (e.g., decreased) insulin levels (e.g., decreased insulin levels in the brain) are associated with decreased synthesis and levels of cholesterol and other sterols (including cholesterol precursors) in the central nervous system (CNS) or brain (e.g., in the hypothalamus and other pertinent areas of the brain, including within synapses). Data presented herein also support that the observed decrease in cholesterol and/or other sterols can promote detrimental or undesirable physiological changes in neural function. Further, the present disclosure demonstrates that increased synthesis and/or levels of cholesterol (and other sterols) can be promoted or restored using insulin.

Accordingly, the present disclosure provides, inter alia, compositions and methods for increasing synthesis and/or levels of cholesterol (and other sterols) in the CNS or in the brain of subjects in need thereof (e.g., in subjects with decreased levels of cholesterol in their CNS or brain (e.g., diabetics and/or untreated/insufficiently treated diabetics)) by increasing the levels of insulin and/or insulin analogues (e.g., insulin and/or insulin analogues that can enter the central nervous system) in the CNS or in the brain of the subject.

The present disclosure also provides that the observed reduction in cholesterol synthesis and/or levels in the CNS or brain correspond with a decrease in the expression of the major transcriptional regulator of cholesterol metabolism, SREBP-2, and its downstream targets or genes in the hypothalamus and other areas of the brain. Further, the data suggest that a decrease in the levels of insulin in the brain directly contribute to decreased SREBP-2 expression. Accordingly, compositions and methods that increase the levels of insulin in the brain can be used to increase SREBP-2 expression in the hypothalamus and other areas of the brain and thereby increase cholesterol expression and/or levels.

The brain is the most cholesterol-rich organ in the body, most of which comes from in situ synthesis (Dietschy and Turley, Curr. Opin. Lipidol., 12:105-112 (2001)). It is generally accepted that because cholesterol is essential for synaptogenesis and synapse function, altered cholesterol biosynthesis can lead to altered brain or neural function. For example, it has been shown that pharmacological depletion of cholesterol from lipid rafts in cultured neuronal cells leads to gradual loss of synapses (Hering et al., J. Neurosci., 23:3262-3271 (2003)). Depletion of cholesterol also has been shown to block the biogenesis of synaptic vesicles (Rohrbough and Broadie, Nat. Rev. Neurosci., 6:139-150 (2005); Thiele et al., Nat. Cell. Biol., 2:42-49 (2000)), and disrupt SNARE clusters leading to decreased neurotransmitter release (Chamberlain et al., Proc. Natl. Acad. Sci. USA, 98:5619-5624 (2001); Lang et al., EMBO J., 20:2202-2213 (2001)).

A number of neuronal abnormalities are reported in mouse models of diabetes, including alterations in learning, memory, synaptic plasticity, and glutamatergic neurotransmission (Biessels and Gispen, Neurobiol Aging 26:Suppl 1, 36-41 (2005)). For example, mice with heterozygous knockout of the insulin receptor exhibit impairment in object recognition (Das et al., 2005). It was previously believed, however, that such abnormalities were caused by hyperglycemia and not altered neuronal insulin levels.

### Therapeutic Agents

Therapeutic agents useful herein include any compound(s) that can enter or that can be modified to enter the CNS or brain (e.g., any compound that can cross the blood brain barrier (BBB)) in an amount and/or for a time sufficient to: (i) increase the expression and/or activity (e.g. transcriptional activity) of SREBP-2 in the CNS or brain of a subject; (ii) increase insulin levels and/or insulin activity in the CNS or brain of a subject; (iii) increase cholesterol synthesis and/or levels in the CNS or brain of a subject; or any combination of (i), (ii) and/or (iii). In some embodiments, the therapeutic agent can be an agent that does not alter plasma glucose levels.

Insulin is reported to cross the blood brain barrier (Cashion et al., Horm. Behav., 30:280-6 (1996); Banks et al., Peptides, 18:1423-9 (1997); Banks et al., Peptides, 18:1257-62 (1997); and Banks et al., Peptides, 18:1577-84 (1997)). Accordingly, in some embodiments, therapeutic agents can include insulin (e.g., porcine insulin, bovine insulin, human insulin, and/or recombinant insulin (e.g., recombinant human insulin)) and/or insulin analogues, including any commercially available insulins and insulin analogues. For example, therapeutic agents can include, but are not limited to, one or more (including all) of regular insulin, insulin Glargine (marketed as Lantus® by Sanofi Aventis), Humulin (Eli Lilly), lispro insulin (Humalog (Eli Lilly)), insulin aspart (NovoLog), Samilente insulin, NPH, lente insulin, Lantus, (Aventis Pharma), Humulin UL Humulin 50/50 (Eli Lilly), Humulin UL (Eli Lilly), Humulin L (Eli Lilly), Humulin R (Eli Lilly), Humulin NPH (Eli Lilly), Humalog Mix25 (Eli Lilly), Humulin 30/70 (Eli Lilly), Humulin 50/50 (Eli Lilly), Ultratard (Norvo Nordisk), Monotard (Novo Nordisk), NovoRapid (Norvo Nordisk), Actrapid (Norvo Nordisk), Protaphane (Norvo Nordisk), Novomix 30 (Norvo Nordisk), Mixtard 30/70 (Norvo Nordisk), Mixtard 50/50 (Norvo Nordisk), Mixtard 20/80 (Norvo Nordisk), and Levemir (Norvo Nordisk), and analogues or modified forms thereof.

Inhalable insulin products include VIAject™ (Biodel Inc.); AERx Insulin Diabetes Management System (Novo Nordisk); QDose inhaled insulin (QDose); Technosphere® Insulin System (MannKind); and Oral-Lyn™ (Generex).

Alternatively or in addition, therapeutic agents can include nucleic acids that increase the expression and/or activity of SREBP-2 in neurons and/or glial cells. For example, such nucleic acids can include naked DNAs and expression constructs (e.g., viral and non-viral expression constructs) that include a SREBP-2 nucleic acid sequence (e.g., NCBI accession no. EF640983.1) or that include a nucleic acid sequence that encodes SREBP-2 protein (e.g., NCBI accession number ABR68260.1) or an active fragment thereof. In some embodiments, useful nucleic acid sequences can have 50%, 60%, 70%, 80%, 85%, 90%, 95%, 98%, 99%, or 100% sequence identity to NCBI accession no. EF640983.1. Therapeutic agents can also include SREBP-2 peptides (e.g., ABR68260.1). In some embodiments, useful amino acid sequences can have 50%, 60%, 70%, 80%, 85%, 90%, 95%, 98%, 99%, or 100% sequence identity to NCBI accession no. ABR68260.1.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The determination of percent identity between two amino acid sequences is accomplished using the BLAST 2.0 program. Sequence comparison is performed using an ungapped alignment and using the default parameters (Blossom 62 matrix, gap existence cost of 11, per residue gapped cost of 1, and a lambda ratio of 0.85). The mathematical algorithm used in BLAST programs is described in Altschul et al. (Nucleic Acids Res. 25:3389-3402, 1997). Useful peptide can also include conservative amino acid substitutions. Conservative amino acid substitutions are known in the art.

In some embodiments, useful peptides can include modified peptides that possess at least a portion of the activity (e.g., biological activity) of the unmodified peptide. For example, modified peptides can retain 50%, 60%, 70%, 80%, 85%, 90%, 95%, 98%, 99%, or 100% of the activity (e.g., biological activity) of unmodified peptide.

Useful proteins or peptides can include fusion proteins comprising a SREBP-2 peptide in combination with a moiety that increase stability of the fusion protein in vivo (e.g., polyethelene glycol (PEG)) and/or that increases transport of the fusion protein across the BBB.

Naked DNA constructs and the therapeutic use of such constructs are well known to those of skill in the art (see, e.g., Chiarella et al., Recent Patents Anti-Infect. Drug Disc., 3:93-101, 2008; Gray et al., Expert Opin. Biol. Ther., 8:911-922, 2008; Melman et al., Hum. Gene Ther., 17:1165-1176, 2008). Typically, naked DNA constructs include one or more therapeutic nucleic acids and a promoter sequence. A naked DNA construct can be a DNA vector, commonly referred to as pDNA. Naked DNA typically do not incorporate into chromosomal DNA. Generally, naked DNA constructs do not require, or are not used in conjunction with, the presence of lipids, polymers, or viral proteins. Such constructs may also include one or more of the non-therapeutic components described herein.

DNA vectors are known in the art and typically are circular double stranded DNA molecules. DNA vectors usually range in size from three to five kilo-base pairs (e.g., including inserted therapeutic nucleic acids). Like naked DNA, DNA vectors can be used to deliver and express one or more therapeutic proteins in target cells. DNA vectors do not incorporate into chromosomal DNA.

Generally, DNA vectors include at least one promoter sequence that allows for replication in a target cell. Uptake of a DNA vector may be facilitated (e.g., improved) by combining the DNA vector with, for example, a cationic lipid, and forming a DNA complex.

Also useful are viral vectors, which are also well known to those of skill in the art. Typically, viral vectors are double stranded circular DNA molecules that are derived from a virus. Viral vectors are typically larger in size than naked DNA and DNA vector constructs and have a greater capacity for the introduction of foreign (i.e., not virally encoded) genes. Like naked DNA and DNA vectors, viral vectors can be used to deliver and express one or more therapeutic nucleic acids in target cells. Unlike naked DNA and DNA vectors, certain viral vectors stably incorporate themselves into chromosomal DNA.

Typically, viral vectors include at least one promoter sequence that allows for replication of one or more vector encoded nucleic acids, e.g., a therapeutic nucleic acid, in a host cell. Viral vectors may optionally include one or more non-therapeutic components described herein. Advantageously, uptake of a viral vector into a target cell does not require additional components, e.g., cationic lipids. Rather, viral vectors transfect or infect cells directly upon contact with a target cell.

The approaches described herein include the use of retroviral vectors, adenovirus-derived vectors, and/or adeno-associated viral vectors as recombinant gene delivery systems for the transfer of exogenous genes in vivo, particularly into humans. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals.

The genome of an adenovirus can be manipulated such that it encodes and expresses a gene product of interest but is inactivated in terms of its ability to replicate in a normal lytic viral life cycle. See, for example, Berkner et al., BioTechniques 6:616, 1988; Rosenfeld et al., Science 252:431-434, 1991; and Rosenfeld et al. Cell 68:143-155, 1992. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham, J. Virol., 57:267, 1986).

Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129, 1992). It is also one of the few viruses that may integrate its DNA into non-dividing cells, and exhibits a high frequency of stable integration (see for example Flotte et al., Am. J. Respir. Cell. Mol. Biol. 7:349-356, 1992; Samulski et al., J. Virol., 63:3822-3828, 1989; and McLaughlin et al., J. Virol., 62:1963-1973, 1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260, 1985 can be used to introduce DNA into cells. Skilled practitioners will appreciate that the use of any number of viral vectors in the presently described methods is possible.

All the molecular biological techniques required to generate an expression construct described herein are standard techniques that will be appreciated by one of skill in the art. Detailed methods may also be found, e.g., Current Protocols in Molecular Biology, Ausubel et al. (eds.) Greene Publishing Associates, (1989), Sections 9.10 9.14 and other standard laboratory manuals. DNA encoding altered-catenin can be generated using, e.g., site directed mutagenesis techniques.

Therapeutic agents can also include small molecules that increase (e.g., specifically increase) the expression and/or activity of SREBP-2.

Pharmaceutical Compositions

In some embodiments, one or more therapeutic agents can be formulated as a pharmaceutical composition. Pharmaceutical compositions containing one or more therapeutic agents can be formulated according to the intended method of administration.

Pharmaceutical compositions containing one or more therapeutic agents can be formulated in a conventional manner using one or more physiologically acceptable carriers or excipients. The nature of the pharmaceutical compositions for administration is dependent on the mode of administration and can readily be determined by one of ordinary skill in the art. In addition, methods for making such formulations are well known and can be found in, for example, Remington's Pharmaceutical Sciences, 18th Ed., Gennaro, ed., Mack Publishing Co., Easton, Pa., 1990. In some embodiments, the pharmaceutical composition is sterile or sterilizable.

The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol.

In some embodiments, the compositions can be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the composition may be in powder form for constitution with a suitable vehicle, for example, sterile pyrogen-free water, before use.

In addition to the formulations described previously, the compositions can also be formulated as a depot preparation. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions can also take the form of tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

Methods for enhancing delivery of drugs to the brain are also known in the art, see, e.g., Ulbrich et al., “Targeting the insulin receptor: nanoparticles for drug delivery across the blood-brain barrier (BBB)”, J Drug Target. 2010 Apr. 13. [Epub ahead of print]; Pardridge, J Drug Target. 2010 April; 18(3):157-67; and Patel et al., CNS Drugs. 2009; 23(1):35-58.

The methods herein contemplate administration of an effective amount of compound or compound composition to achieve the desired or stated effect. Toxicity and therapeutic efficacy of the compounds and pharmaceutical compositions described herein can be determined by standard pharmaceutical procedures, using either cells in culture or experimental animals to determine the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED50 with little or no toxicity, and with little or no adverse effect on a human's ability to hear. The dosage may vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC50 (that is, the concentration of the test compound which achieves a half-maximal inhibition of symptoms) as determined in cell culture. Such information can be used to more accurately determine useful doses in humans.

The formulations and routes of administration can be tailored to the disease or disorder being treated, and for the specific human being treated. A subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

Generally the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., Chapter 27 In: Remington's Pharmaceutical Sciences, 18th Ed., Gennaro, ed., Mack Publishing Co., Easton, Pa., 1990). Generally, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3, In: Goodman & Gilman's “The Pharmacological Basis of Therapeutics”, 9th Ed., Hardman et al., eds., McGraw-Hill, New York, N.Y., 1996).

### Methods of Treatment

In some embodiments, the present disclosure includes compositions and methods for treating nervous dysfunction (e.g., Alzheimer's or dementia), including nervous disorders or associated with diabetes (e.g., diabetic brain dysfunctions, including depression, mood and behavioral changes, and diabetic neuropathies). For example, the present disclosure includes compositions and methods for treating a nervous disorder (e.g., diabetic neuropathy) in a diabetic subject in which the subject has reduced cholesterol levels and/or reduced SREBP-2 expression and/or activity in their CNS, e.g., associated with decreased insulin levels in the CNS. For example, the nervous disorder can include diabetic neuropathy. Exemplary neuropathies include CNS disorders, cognitive dysfunctions (cognitive disorders), memory losses, Alzheimer's and dementia.

As used herein, “treatment” means any manner in which one or more of the symptoms of the nervous dysfunction or disorder are ameliorated or otherwise beneficially altered. As used herein, amelioration of the symptoms of the disorder refers to any lessening, whether permanent or temporary, lasting or transient that can be attributed to or associated with treatment by the compositions and methods of the present invention.

In certain embodiments, a subject having a neuropathy and being treated as described herein has not been administered (e.g., has not received) insulin or an insulin analog prior to the administration of insulin or an insulin analog for treating the neuropathy. In certain embodiments a subject having a neuropathy and being treated as described herein has not been administered an insulin or an insulin analog, e.g., wherein the insulin or insulin analog crosses the Blood Brain Barrier (BBB), e.g., efficaciously crosses the BBB, prior to the beginning of the neuropathy treatment. In certain embodiments, a subject having a neuropathy and being treated as described herein is not being administered insulin or an insulin analog, e.g., wherein the insulin or insulin analog crosses the BBB (e.g., efficaciously crosses the BBB) for treating a condition that is not a neuropathy, at the time of administration of insulin or insulin analog for treating a neuropathy.

In certain embodiments, insulin or an insulin analog, wherein the insulin or insulin analog crosses the BBB (e.g., efficaciously crosses the BBB) is being administered to a subject having a neuropathy with the purpose of treating the neuropathy. A subject having a neuropathy may be treated with insulin or an insulin analog, wherein the sole purpose of the administration is for treating the neuropathy.

In certain embodiments, a subject having a neuropathy and being treated as described herein was, or simultaneously is, being administered insulin or an insulin analog, e.g., wherein the insulin or insulin analog crosses the BBB, for treating a different indication. For example, in certain embodiments, a subject was, or simultaneously is being administered insulin or an insulin analog for the treatment of type 1 or type 2 diabetes, insulin resistance or the metabolic syndrome.

In certain embodiments, a subject having a neuropathy and being treated as described herein was administered insulin or an insulin analog prior to administration of insulin or an insulin analog for treating the neuropathy, wherein the dose of insulin or insulin analog for treating the neuropathy is different from the dose of insulin or insulin analog that the subject received prior to administration of insulin or insulin analog for treating the neuropathy. The dose of insulin or insulin analog may be changed (e.g., switched) to the dose of insulin or insulin analog that is administered for treating the neuropathy. The dose of insulin or insulin analog for treating the neuropathy may be higher than the dose of insulin or insulin analog that was administered to the subject prior to administration of insulin or insulin analog for treating the neuropathy, and the subject may be administered the higher dose of insulin or insulin analog for treating the neuropathy.

In certain embodiments, a subject having a neuropathy and being treated as described herein was administered insulin or an insulin analog prior to administration of insulin or an insulin analog for treating the neuropathy, wherein the regimen of insulin or insulin analog administration for treating the neuropathy is different from the regimen of administration of insulin or insulin analog that the subject received prior to administration of insulin or an insulin analog for treating the neuropathy. The regimen of administration of insulin or insulin analog may be changed to that for treating the neuropathy. For example, the insulin or insulin analog is administered more frequently for the treatment of neuropathy than administration of insulin or insulin analog prior to administration of insulin or insulin analog for treating the neuropathy. In some embodiments, the insulin or insulin analog is administered more frequently and at a higher dose than the insulin or insulin analog was administered to the subject prior to the start of the administration of insulin or insulin analog for the treatment of the neuropathy.

In certain embodiments, a subject having a neuropathy and being treated as described herein was administered insulin or an insulin analog prior to administration of insulin or an insulin analog for treating the neuropathy, wherein insulin or an insulin analog was administered essentially continuously, e.g., with an insulin pump, prior to starting the neuropathy treatment, and the subject is further administered a second treatment with insulin or an insulin analog. The second treatment, i.e., for the neuropathy, may comprise discrete administrations of insulin or an insulin analog. It may the be same insulin or insulin analog or a different insulin or insulin analog relative to that used prior to the neuropathy treatment. For example, it may be an insulin or insulin analog that effectively reaches the brain.

In certain embodiments, a subject who is being treated for neuropathy as described herein was being administered insulin or an insulin analog prior to administration of insulin or an insulin analog for treating the neuropathy, wherein the insulin or insulin analog that is being administered for treating the neuropathy is different from the insulin or insulin analog that the subject received prior to administration of insulin or an insulin analog for treating the neuropathy. The insulin or analog may be changed to that for treating the neuropathy. In certain embodiments, the insulin or insulin analog that was being administered prior to administration of insulin or insulin analog for treating a neuropathy was not a form of insulin or analog of insulin with effective crossing of the BBB, and the insulin or insulin analog that is being administered for treating the neuropathy is a form of insulin or insulin analog that more effectively crosses the BBB relative to the insulin or insulin analog that was administered to the subject prior to administration of insulin or insulin analog for treating the neuropathy. In certain embodiments, the form of insulin or insulin analog that was being administered to the subject prior to administration of insulin or an insulin analog for treating a neuropathy, is switched to the form for treating a neuropathy, e.g., a form that crosses the BBB more efficaciously than the form that was administered to the subject prior to the start of the neuropathy treatment with an insulin or insulin analog. In certain embodiments, the insulin or insulin analog that was being administered prior to administration of insulin or an insulin analog for treating a neuropathy was not, e.g., a form of insulin or insulin analog that is administered to the head, such as an inhalable, nasal or oral form of insulin or insulin analog, and the insulin or insulin analog that is being administered for treating a neuropathy is a form of insulin or insulin analog that is administered to the head, e.g., an inhalable, nasal or oral form of insulin or insulin analog. In certain embodiments, administration of a non-inhalable, non-nasal or non-oral form of insulin or an insulin analog is switched to administration of an inhalable, nasal or oral form of insulin or an insulin analog, for the treatment of the neuropathy. A “nasal” insulin or insulin analog is an insulin or insulin analog that is administered to the nasal cavity of a subject, e.g., with a spray or drops. An “oral” insulin or insulin analog is an insulin or insulin analog that is administered to the oral cavity of a subject.

In certain embodiments, a subject who is being treated for neuropathy as described herein was being administered insulin or an insulin analog prior to administration of insulin or insulin analog for treating the neuropathy, and the insulin or insulin analog that is being administered for treating the neuropathy is different from the insulin or insulin analog that the subject received prior to administration of insulin or an insulin analog for treating the neuropathy. In certain embodiments, the subject is being administered both (i) the insulin or insulin analog that was administered prior to administration of insulin or insulin analog for treating the neuropathy and (ii) the insulin or analog for treating the neuropathy. For example, the subject may be administered a form of insulin or insulin analog that does not effectively cross the BBB prior to, and during, administration of insulin or insulin analog for treating a neuropathy, and the subject is further being administered a form of insulin or insulin analog that crosses the BBB more effectively than the form of insulin or insulin analog that was being administered to the subject prior to administration of insulin or insulin analog for the treatment of the neuropathy. The subject may being administered a non-inhalable form of insulin or insulin analog prior to and during administration of insulin or insulin analog for treating the neuropathy, and the subject is further being administered an inhalable, nasal or oral form of insulin or insulin analog for the treatment of the neuropathy.

Thus, a subject who is being treating for a neuropathy as further described herein may receive two types of insulin or insulin analogs: (i) a first insulin or insulin analog that favors activity in the brain (for treating the neuropathy) and a second insulin or insulin analog that favors activity outside of the brain (for treating any other condition, such as type 1 or type 2 diabetes or precursor or related condition thereof). The dose, regimen, formulation and/or type of administration of each of these two insulin or insulin analog drugs (or compositions, e.g., pharmaceutical compositions) may be the same or different. For example, the concentration of one or the other may be higher; one or the other may be administered more or less frequently; one may be administered essentially continuously, while the other is administered as discrete doses; one may be an inhalable, nasal or oral form, while the other is a non-inhalable, non-nasal or non-oral form, respectively. On the other hand, e.g., as further described above, in certain embodiments, an insulin or insulin analog was administered to a subject for a different purpose than for treating a neuropathy, and the insulin or insulin analog is switched to a different insulin or insulin analog, a different dose, a different regimen, a different formulation, and/or a different type of administration, for the treatment of the neuropathy. The two treatments may also be overlapping for a certain period of time. For example, administration to a subject of an insulin or insulin analog that does not effectively cross the BBB is substituted for an insulin or insulin analog that more effectively crosses the BBB. The insulin or insulin analog may also be an insulin or insulin analog that is also effective for the other disorder that the subject may have, e.g., the insulin or insulin analog is effective in crossing the BBB and in treating diabetes.

An insulin or insulin analog that crosses the BBB may be an insulin or insulin analog that is administered to the head of a subject, e.g., orally or intranasally. Intranasal administration includes inhalation, which may occur with the use of a spray.

Exemplary oral, nasal, and inhalable forms of insulin or insulin analogs include, e.g., Exu-bera® and Ora-Lyn®. Inhaled insulin includes systemic delivery of insulin via the pulmonary route of administration made by inhalation of, e.g., a spray dried powder. Buccal insulin delivery includes, e.g., systemic insulin delivery via the absorption of insulin in the oral cavity, and may be made by spraying a liquid insulin composition directly into the oral cavity. An exemplary oral insulin composition is a concentrated insulin solution, e.g., described in WO2008132229, wherein the insulin concentration is, e.g., above 12, 15, 20, 30, 40, 50 or 60 mM. The insulin may be human or non-human insulin, e.g., porcine insulin.

Exemplary insulin analogs that may be used include the following: an insulin analogue wherein the amino acid residue in position B28 of insulin is Pro, Asp, Lys, Leu, VaI, or Ala and the amino acid residue in position B29 is Lys or Pro and optionally the amino acid residue in position B30 is deleted;

des(B28-B30) human insulin, des(B27) human insulin or des(B30) human insulin; an insulin analogue wherein the amino acid residue in position B3 is Lys and the amino acid residue in position B29 is GIu or Asp;
an insulin analogue wherein the amino acid in position A14 is selected from the group consisting of Lys, GIu, Arg, Asp, Pro and His, the amino acid in position B25 is His and which optionally further comprises one or more additional mutations;
an insulin analogue wherein

the amino acid in position A8 is His and/or the amino acid in position A12 is GIu or Asp and/or the amino acid in position A13 is His, Asn, GIu or Asp and/or the amino acid in position A14 is Asn, GIn, GIu, Arg, Asp, GIy or His and/or the amino acid in position A15 is GIu or Asp; and

the amino acid in position B1 is GIu and/or the amino acid in position B16 is GIu or His and or the amino acid in position B25 is His and/or the amino acid in position B26 is His, GIy, Asp or Thr and/or the amino acid in position B27 is His, GIu, Lys, GIy or Arg and/or the amino acid in position B28 is His, GIy or Asp; and which optionally further comprises one or more additional mutations; and

an insulin analogue wherein the amino acid in position A14 is selected from the group consisting of Lys, GIu, Arg, Asp, Pro and His; and the B-chain of the insulin analogue comprises at least two mutations relative to the parent insulin, wherein two or more mutations are in the form of deletions of the amino acids in positions B27, B28, B29 and B30, or a combination of a deletion of the amino acid in position B30 and a substitution of an amino acid selected from the amino acid substitutions in position: B25 to His, B26 to GIy or GIu, B27 to GIy or Lys and B28 to Asp, His, GIy, Lys or GIu.

Exemplary human insulin analogs that may be used also include: DesB30 human insulin; AspB28 human insulin; AspB28,DesB30 human insulin; LysB3,GluB29 human insulin; LysB28,ProB29 human insulin; GluA14,HisB25 human insulin; HisA14,HisB25 human insulin; GluA14,HisB25,DesB30 human insulin; HisA14,HisB25,DesB30 human insulin; GluA14,HisB25,desB27,desB28,desB29,desB30 human insulin; GluA14,HisB25,GluB27,desB30 human insulin; GluA14,HisB16,HisB25,desB30 human insulin; HisA14,HisB16,HisB25,desB30 human insulin; HisA8,GluA14,HisB25,GluB27,desB30 human insulin; HisA8,GluA14, GIuBI, GluB16,HisB25,GluB27,desB30 human insulin; and HisA8,GluA14,GluB16,HisB25,desB30 human insulin.

Any other insulin analogs or homologs or variants may be used, provided that it increases cholesterol synthesis in the brain.

Subject Selection

The term “subject” is used throughout the specification to describe an animal, human or non-human, to whom treatment according to the methods of the present invention is provided.

The methods disclosed herein can include selecting a subject for treatment. For example, a subject can be selected if the subject has or is at risk for developing a neurological condition or dysfunction. For example, a subject can be selected if the subject has or is a risk for developing neuropathy, e.g., diabetic neuropathy, including peripheral neuropathy, autonomic neuropathy, proximal neuropathy, focal neuropathy, diabetic amyotrophy, and mononeuropathy. In some instances, the subject can be identified as a subject with diabetic neuropathy. Alternatively or in addition, the subject can be a subject with one or more symptoms of diabetic neuropathy, including, but not limited to, pain, numbness and/or tingling of extremities, dysesthesia, diarrhea, erectile dysfunction, urinary incontinence (loss of bladder control), impotence, facial, mouth, and eyelid drooping, vision changes, dizziness, muscle weakness, difficulty swallowing, speech impairment, fasciculation (muscle contractions), anorgasmia, burning or electric pain, pain, seizures, and weakness.

A subject in need of therapy described herein may also be a subject who has or is likely to have, or to develop, a neuropathy, such as a CNS disorder, e.g., a cognitive disorder, Alzheimer's disease, dementia, memory losses, or other CNS disorder that is characterized by (or associated with) a reduction in brain cholesterol levels. A subject may be a subject having diabetes, such as type 1 or 2 diabetes. A method may comprise first diagnosing a subject as being in need of a therapy described herein, and then administering the therapy. The status of the disease, e.g., its progression or regression, may be followed during treatment.

In some embodiments, a subject can be selected if the subject has, is at risk of having, or is suspected (e.g., by a health care professional) of having decreased levels or expression of cholesterol in their CNS. Subjects can also be selected is they have decreased levels of SREBP-2 expression or activity in the CNS.

A subject may also be a subject who has or a subject who is likely of developing diabetes, such as type 1 or type 2 diabetes, insulin resistance or the metabolic syndrome or a subject who produces reduced insulin levels relative to a healthy subject. A subject may also be a subject who does not have diabetes, such as type 1 or type 2 diabetes, insulin resistance or the metabolic syndrome.

Routes of Administration

The therapeutic agents and pharmaceutical compositions of this disclosure may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intra-articular, intraarterial, intrasynovial, intrasternal, intrathecal, intralesional and intracranial injection or infusion techniques. Alternatively or in addition, the present invention may be administered according to any of the Food and Drug Administration approved methods, for example, as described in CDER Data Standards Manual, version number 004 (which is available at fda.give/cder/dsm/DRG/drg00301.htm). Where application over a period of time is advisable or desirable, the compositions of the invention can be placed in sustained released formulations or implantable devices (e.g., an implantable pump).

Subject Evaluation

The methods can also include monitoring or evaluating the subject during and after treatment to determine the efficacy of the treatment, and, if necessary, adjusting treatment (e.g., by altering the composition, by increasing the dose of a single administration of the composition, by increasing the number of doses of the composition administered per day, and/or by increasing the number of days the composition is administered) to improve efficacy. Monitoring or evaluating the subject can include identifying a suitable marker of disease prior to commencing treatment and optionally recording the marker, and comparing the identified or recorded marker to the same marker during and/or after treatment. Suitable markers can include one or more symptoms of the subject's disease. In some instances, the marker can include cholesterol levels and/or SREBP-2 expression or activity in the subject's CNS (methods for assessing cholesterol levels and SREBP-2 expression or activity are known in the art and are disclosed herein). Adjustment of treatment would be recommended where the marker is a symptom of disease and comparison of the marker during or after treatment with the marker prior to treatment revealed either no change in the marker or an increase in the marker. Similarly, adjustment of treatment would be recommended where the marker is cholesterol level or SRBEP-2 expression or activity and where no increase in the marker is observed. Conversely, adjustment of treatment may not be required using the same markers where an increase in the marker is observed.

### Screening Methods

The present disclosure includes methods for identifying therapeutic agents that can enter or that can be modified to enter the CNS or brain (e.g., any compound that can cross the blood brain barrier (BBB)) in an amount and/or for a time sufficient to: (i) increase the expression and/or activity (e.g. transcriptional activity) of SREBP-2 in the CNS or brain of a subject; (ii) increase insulin levels and/or insulin activity in the CNS or brain of a subject; (iii) increase cholesterol synthesis and/or levels in the CNS or brain of a subject; or any combination of (i), (ii) and/or (iii), by screening libraries or collections of compounds or candidate compounds. In some embodiments, the therapeutic agent can be an agent that does not alter plasma glucose levels. Suitable compounds for screening can include peptides, nucleic acids and nucleic acid containing compounds, antibodies and antibody fragments, small molecules, hormones and hormone analogues, insulins and insulin analogues.

Screens can be high throughput and can include establishing a reporter cell line, e.g., a cell line that includes a genetic reporter that is activated by SREBP-2 transcriptional activity. Compounds that increase expression or detection of the marker can then be evaluated for their ability to cross the BBB. Such methods can include administering the compound to a suitable animal model by peripheral injection and evaluating the level of cholesterol expression in the CNS of the animal. In some instances, the methods can include comparing the level of cholesterol expression measured following treatment with the test compound to the level of cholesterol expression in the animal following peripheral administration of insulin to the animal.

Other methods include measuring cerebrospinal fluid levels of the compound, e.g., of an insulin analog, versus plasma levels of the compound, and/or measuring insulin signaling (and/or SREBP-2 transcriptional activity) in the CNS following administration, e.g., peripheral administration, of a compound. The methods can also include determining the ability of a compound, e.g., an insulin analog, to acutely stimulate signaling such as insulin receptor and substrate phosphorylation or Akt phosphorylation following peripheral injection; and/or assaying the ability of a compound, e.g., an insulin analog, to reverse changes in cholesterol metabolism in brain as compared to effects to lower blood glucose.

The same assays could be used to evaluate delivery approaches, such as nasal insulin vs. peripheral insulin injection Methods for performing such screens are known in the art and are disclosed herein. See, e.g., Banks et al., Peptides 31 (2010) 2284-2288;

Henkin et al., Nutrition. 2010 June; 26(6):624-33. Epub 2009 Dec. 22; Vavilala et al., Pediatr Crit Care Med. 2010 May; 11(3):332-8; Hallschmid et al., Diabetologia. 2009 November; 52(11):2264-9. Epub 2009 Aug. 25; Laron, Arch Physiol Biochem. 2009 May; 115(2):112-6; and Hanson and Frey, BMC Neurosci. 2008 Dec. 10; 9 Suppl 3:S5. Either one or both of cell line and animal model screens can be performed.

The invention is further described in the following examples, which do not limit the scope of the invention described in the claims.

### EXAMPLES

### Example 1

### Down Regulation of the Cholesterol Biosynthesis Pathway in Hypothalami of Diabetic Mice

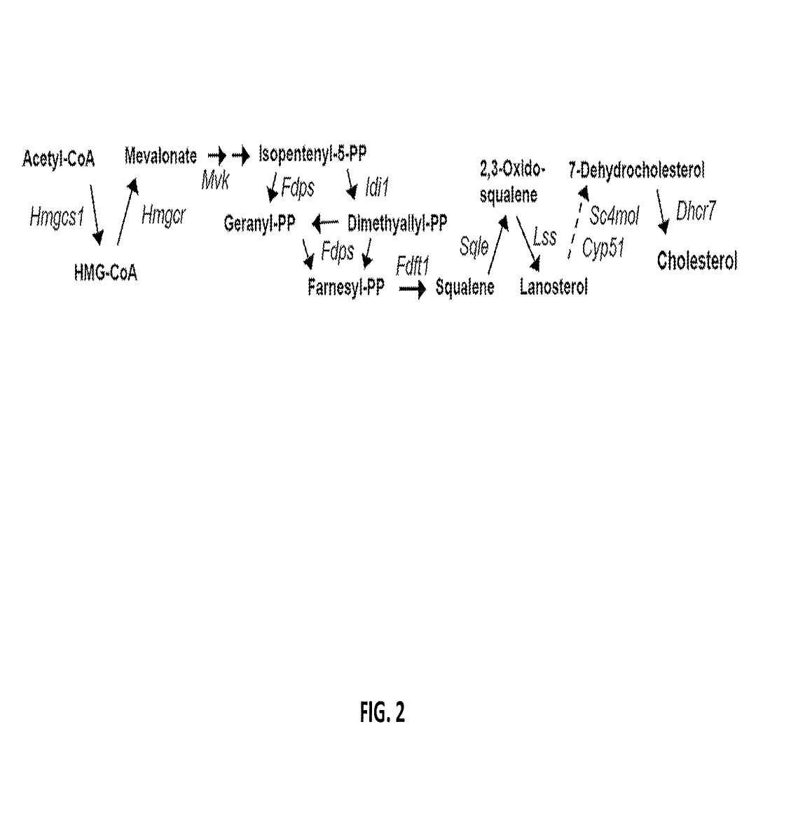

As noted above, almost all cholesterol present in the brain is formed by de novo synthesis, since the blood-brain barrier effectively prevents uptake from the circulation (Björkhem and Meaney, 24:806-815 (2004); Dietschy and Turley, J Lipid Res 45:1375-1397 (2004)). The hypothalamus is a major point of control of the endocrine system, appetite and energy balance (Obici and Rossetti, Endocrinology 144:5172-5178 (2003)). A schematic of the cholesterol-synthesis pathway is shown in FIG. 2.

In an effort to determine how diabetes affects hypothalamic function, oligonucleotide microarrays were used to identify genes differentially expressed in the hypothalamus in the streptozotocin (STZ)-induced diabetic mice (a model of insulin-deficient type 1 diabetes).

C57Bl/6, ob/ob (C57Bl/6 background), db/+ and db/db (C57Bl/Ks background) mice were from Jackson laboratory (Bar Harbor, Me.). NIRKO mice were generated as previously described (Bruning et al., Science, 289:2122-2125 (2001)). NOD mice were provided by Drs. Diane Mathis and Wenyu Jiang (Harvard Medical School). All mice used for experiments were male.

Mice were maintained on a 12-h light/12-h dark cycle and fed a standard mouse chow diet (LabDiet Mouse Diet 9 F, PMI Nutrition International, Brentwood, Mo.). As a diet-induced obesity (DIO) model, C57Bl/6 mice were fed with 60% kcal fat diet (D12492, Research Diet Inc., New Brunswick, N.J.) for 6 months.

Mice were maintained on a 12-h light/12-h dark cycle and fed a standard mouse chow diet (LabDiet Mouse Diet 9 F, PMI Nutrition International, Brentwood, Mo.). As a diet-induced obesity (DIO) model, C57Bl/6 mice were fed with 60% kcal fat diet (D12492, Research Diet Inc., New Brunswick, N.J.) for 6 months.

For STZ-induced diabetes and systemic insulin therapy experiments, 7-week-old C57Bl/6 mice were treated with a single intraperitoneal (i.p.) injection (200 μg/g body weight) of STZ (Sigma). After 2 days, the mice were separated into two groups. Half remained untreated, and the other half were treated with subcutaneous insulin pellets (LinShin, Toronto, Canada) for one week to control blood glucose levels. For phlorizin (PHZ) treatment, 8-week-old C57Bl/6 mice were treated with STZ. PHZ (Sigma) was dissolved in a solution containing 10% ethanol, 15% DMSO, and 75% saline and was injected subcutaneously (0.4 g/kg) twice daily for 10 days starting 8 days after the STZ injection. Control mice were injected with the same volume of vehicle.

Mice were anesthesized with a 2.5% solution of 2:1 mixture of 2,2,2-tribromoethanol and tertiary amyl alcohol (15 μl/g body weight, i.p.). The brain was quickly removed, placed on ice and dissected using a mouse brain matrix (ASI Instruments Inc., Warren, Mich.). All animal studies followed National Institutes of Health guidelines and were approved by the Institutional Animal Care and Use Committees at the Joslin Diabetes Center.

RNA was isolated from 9-week-old male control C57Bl/6 (n=6), STZ-treated C57Bl/6 (n=6), and ob/ob on C57Bl/6 background (n=5) mouse hypothalami. Double-stranded cDNA synthesis was reverse-transcribed from 10 μg of isolated RNA from each hypothalamus by using the SuperScript (Invitrogen) with an oligo(dT) primer containing a T7 RNA polymerase promoter site. Double-stranded cDNA was purified with Phase Lock Gel (Eppendorf). Biotin-labeled cRNA was transcribed by using a BioArray RNA transcript labeling kit (Enzo). A hybridization mixture containing 15 μg of biotinylated cRNA, adjusted for possible carryover of residual total RNA, was prepared and hybridized to mouse Affymetrix GeneChip Mouse Genome 430A 2.0 Arrays. Intensity values were quantified by using MAS 5.0 software (Affymetrix) and analyzed by Gene Set Enrichment Analysis (GSEA) v2.0 software (www.broad.mit.edu/gsea). The heat map shown in FIG. 2 was generated by Gene Set Enrichment Analysis (GSEA).

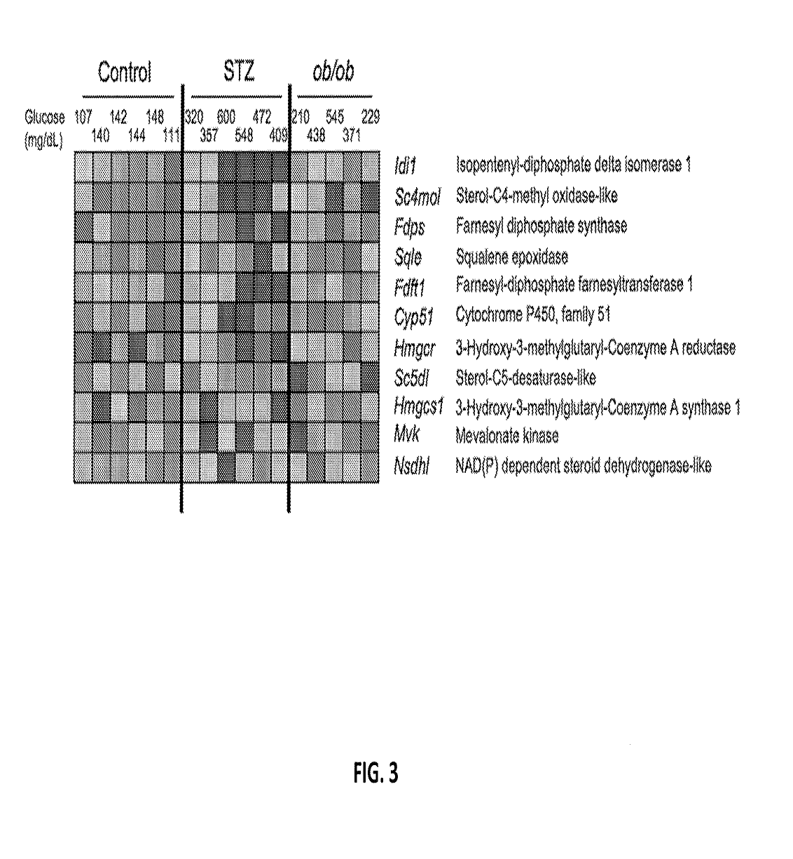

As shown in FIG. 3, GSEA indicated the cholesterol biosynthesis pathway as one of the most highly regulated gene sets in the hypothalamus of the STZ-diabetic mouse, with a broad decrease in expression of multiple cholesterologenic genes.

Confirmation of these microarray data was performed using quantitative real-time PCR (qPCR) and immune blotting.

For qPCR, RNA was isolated using an RNeasy kit (Qiagen). As a template, 1 μg (for tissue) or 0.2 μg (for glia and neurons) of total RNA was reverse-transcribed in 50 μl using a High Capacity cDNA Reverse Transcription Kit (Applied Biosystems) according to the manufacturer's instructions. Three microliters of diluted (1/4) reverse transcription reaction was amplified with specific primers (300 nM each) in a 25 μl PCR with a SYBR Green PCR Master Mix (Applied Biosystems). Analysis of gene expression was done in the ABI PRISM 7000 sequence detector with an initial denaturation at 95° C. for 10 minutes followed by 40 PCR cycles, each cycle consisting of 95° C. for 15 seconds and 60° C. for 1 minute, and SYBR Green fluorescence emissions were monitored after each cycle. For each gene, mRNA expression was calculated relative to Tbp (mouse) or ribosomal 18S (human) expression as an internal control.

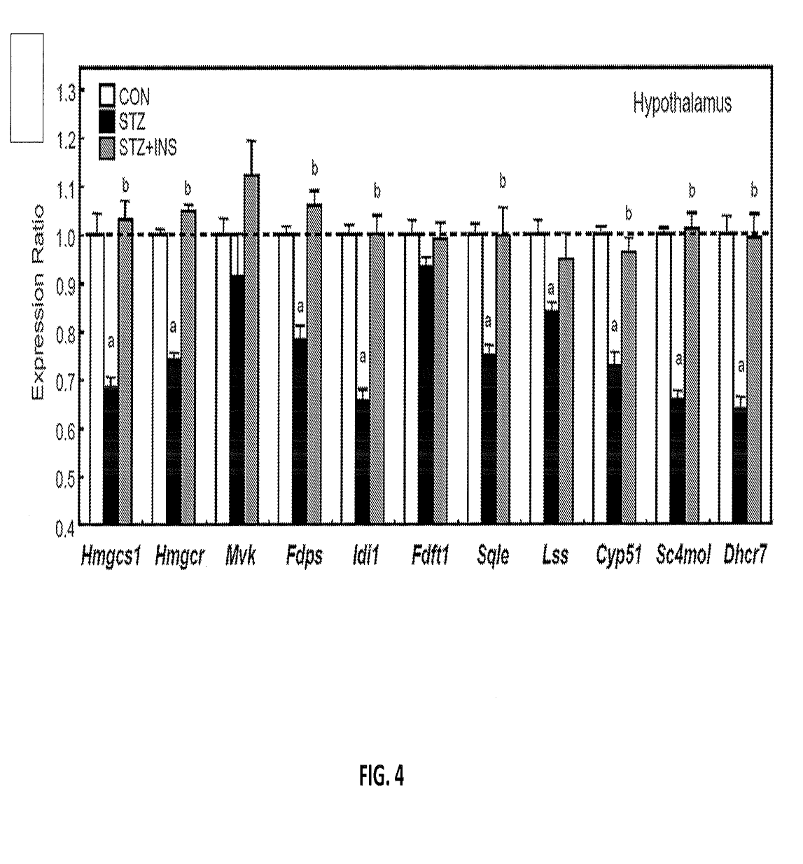

As shown in FIG. 4, qPCR confirmed significant decreases in the majority of the genes encoding enzymes in the pathway producing cholesterol, including a 26% decrease in the rate-limiting enzyme 3-hydroxy-3-methylglutaryl-CoA reductase (Hmgcr) and decreases in mRNA for other cholesterologenic enzymes ranging from 7-36%. In addition, all changes in cholesterologenic genes were reversed by insulin treatment of the diabetic mice.

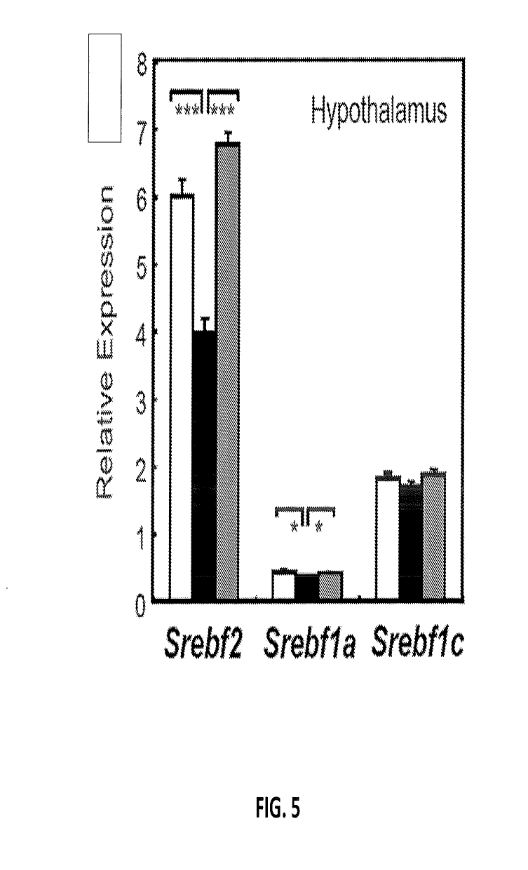

As shown in FIG. 5, also by qPCR, SREBP-2 (Srebf2) was down-regulated by 34% in the STZ-diabetic mice. The expression level of SREBP-1a (Srebf1a) was also slightly, but significantly, reduced in the STZ hypothalamus, whereas SREBP-1c (Srebf1c) expression was unaffected (see FIG. 5).

For immune blots, nuclear and cytoplasmic extracts were prepared using total hypothalamus (about 30 mg) per the manufacturer's directions (NE-PER kit, Pierce). Brain tissue extracts were homogenized in Cell Disruption Buffer (PARIS kit, Ambion). Protein concentrations were measured using a BCA assay (Pierce). Immunoblotting was performed with antibodies against HMGCR (Millipore), FDPS (Abcam), SQLE (ProteinTech, Chicago, Ill.), actin (Santa Cruz), lamin (Cell Signaling Technology), SREBP-1, or SREBP-2 (gifts from Drs. Jay Horton and Guosheng Liang).

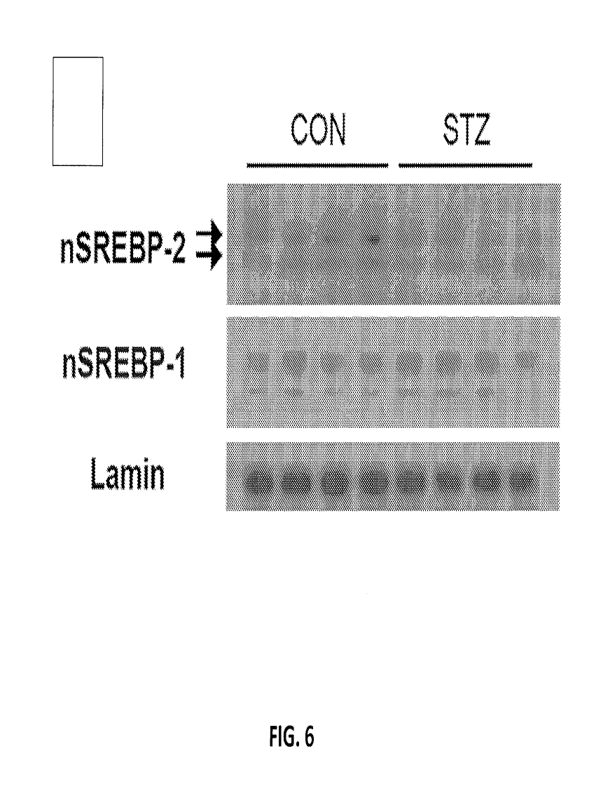

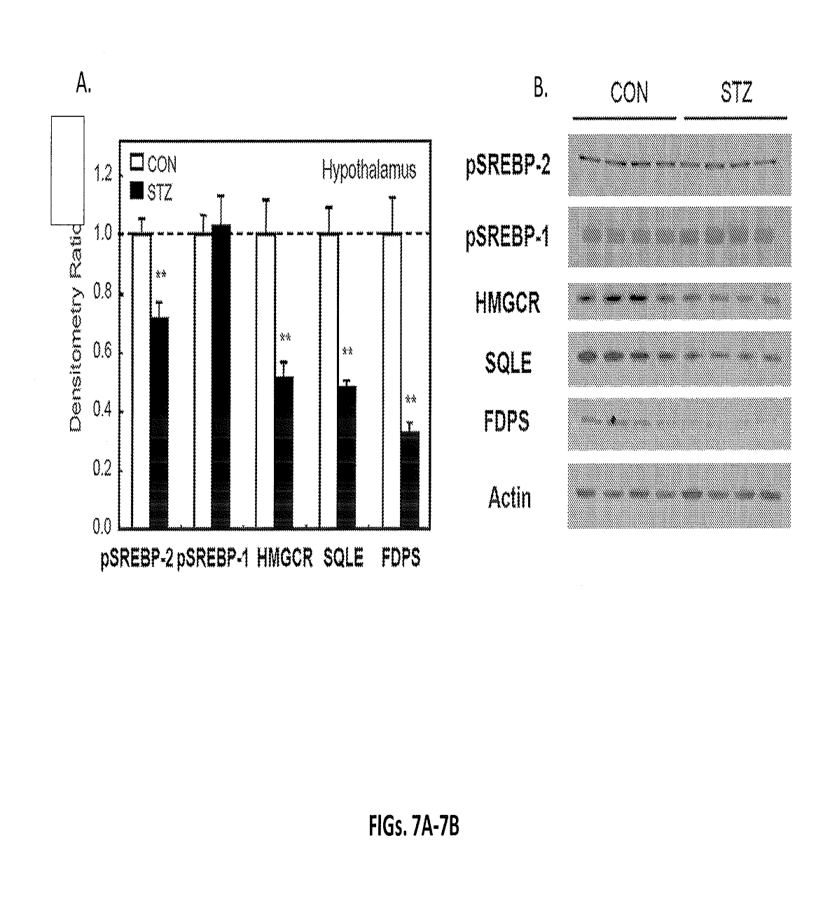

As shown in FIG. 6, Western blotting of hypothalamic extracts revealed that the nuclear mature form of SREBP-2 protein, i.e. transcriptionally active form, was also decreased. As shown in FIGS. 7A and 7B, consistent with mRNA data, protein levels of HMGCR, farnesyl diphosphate synthase (FDPS) and squalene epoxidase (SQLE), as well as those of the cytoplasmic precursor form of SREBP-2 were decreased, as assessed by Western blotting.

Together, these data suggest that the cholesterol biosynthesis pathway is down-regulated in hypothalamic of diabetic mice.

### Example 2

### Cholesterol Synthesis Pathway is Suppressed in Other Diabetes Models and Throughout the Brain

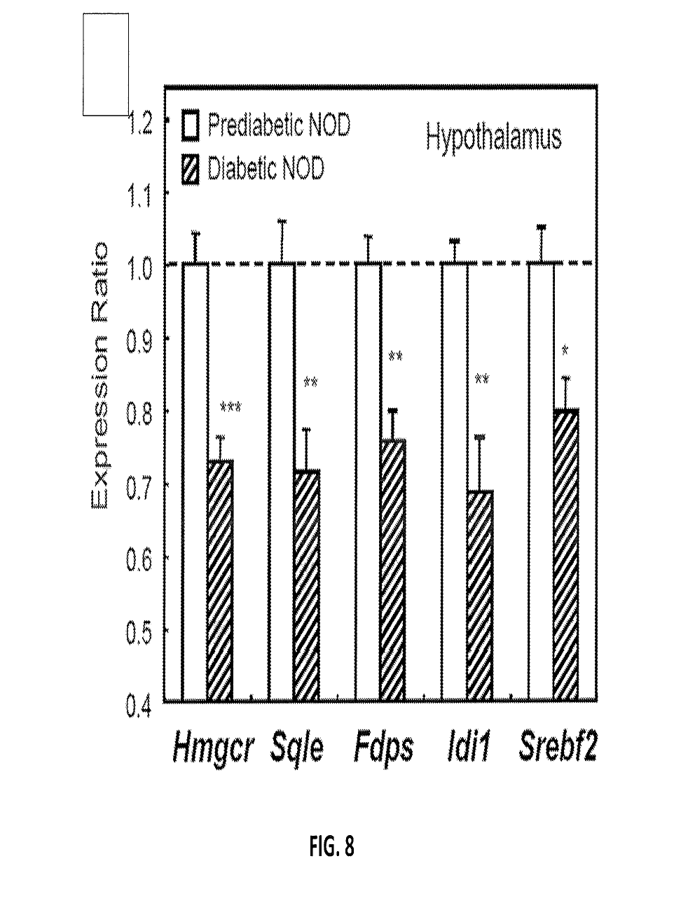

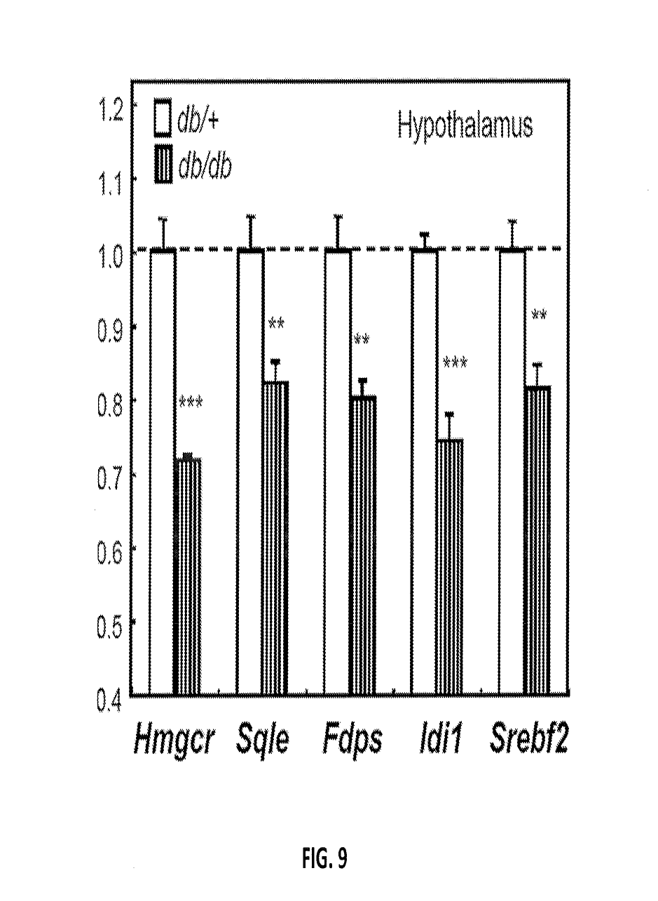

mRNA Levels of cholesterol synthetic enzymes were assessed by qPCR using the methods disclosed in Example 1. As shown in FIGS. 8-12, a reduction in cholesterol synthetic enzymes at the mRNA level was observed in multiple diabetes models in which insulin levels were reduced. Specifically, as shown in FIG. 8, Srebf2 and its downstream genes were down-regulated by ˜30% in the hypothalami from non-obese diabetic (NOD) mice, an autoimmune model of type 1 diabetes. Similar results were also observed in hypothalami from obese, insulin-resistant db/db mice on a C57Bl/Ks background (see FIG. 9), which exhibit a combination of obesity, insulin resistance, and declining insulin levels due to progressive β-cell failure (Uchida et al., Nat. Med., 11:175-182 (2005)).

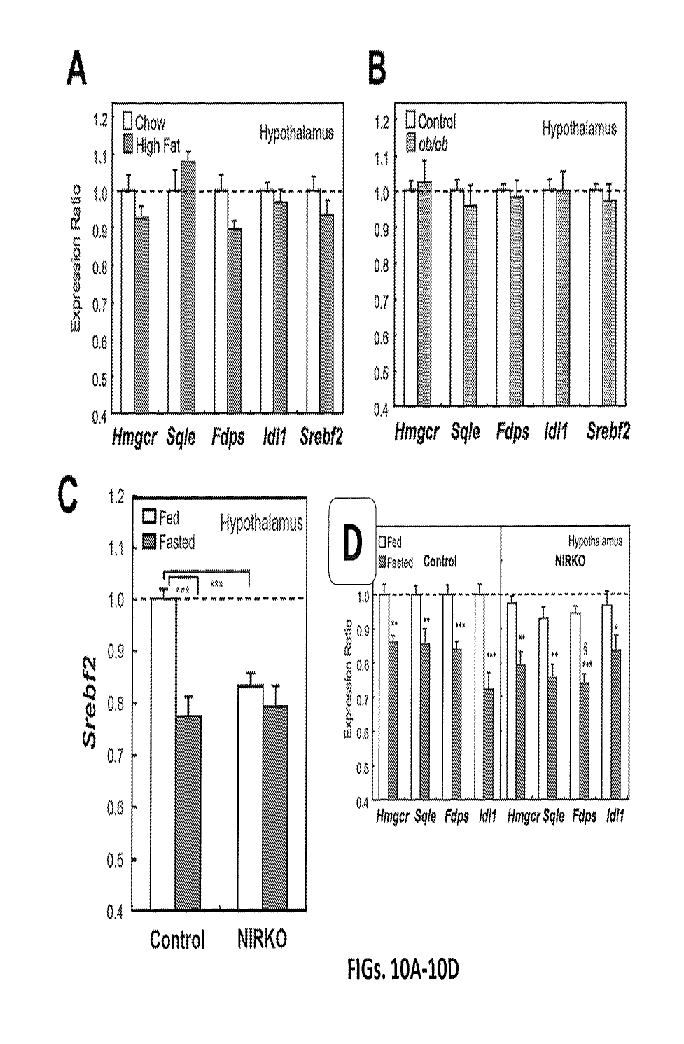

By contrast, hypothalami of mice with dietary-induced (DIO) and genetic obesity (ob/ob), which have insulin resistance without loss of insulin secretion and milder degrees of hyperglycemia, showed no alteration in expression of these genes by microarray analysis (see FIG. 3) or qPCR (see FIGS. 10A-B). These results suggest that absolute or relative decreases in circulating insulin levels and/or the degree of hyperglycemia, but not obesity or systemic insulin resistance, cause suppression of SREBP-2 and cholesterol synthesis pathway in the brain. As shown in FIGS. 10C and 10D, consistent with a role for insulin in SREBP-2 regulation in brain, fasting for 24 hours, which results in a decrease in both insulin and glucose, also caused down-regulation of Srebf2 and cholesterol synthetic genes in mouse hypothalami similar to that seen in STZ-diabetic mice. Srebf2 was also significantly down-regulated in the hypothalami of NIRKO mice with brain-specific insulin receptor knockout (Bruning et al., Science, 289:2122-2125 (2000)), and in this model, there was no further reduction by fasting, consistent with insulin being one of the factors regulating Srebf2 expression in the CNS (see FIG. 10C).

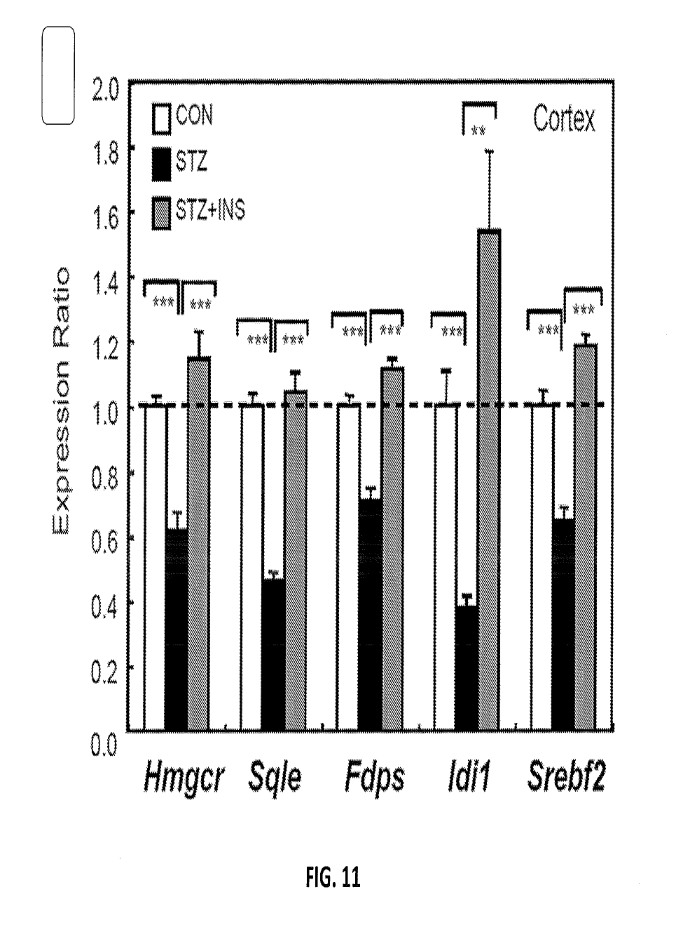

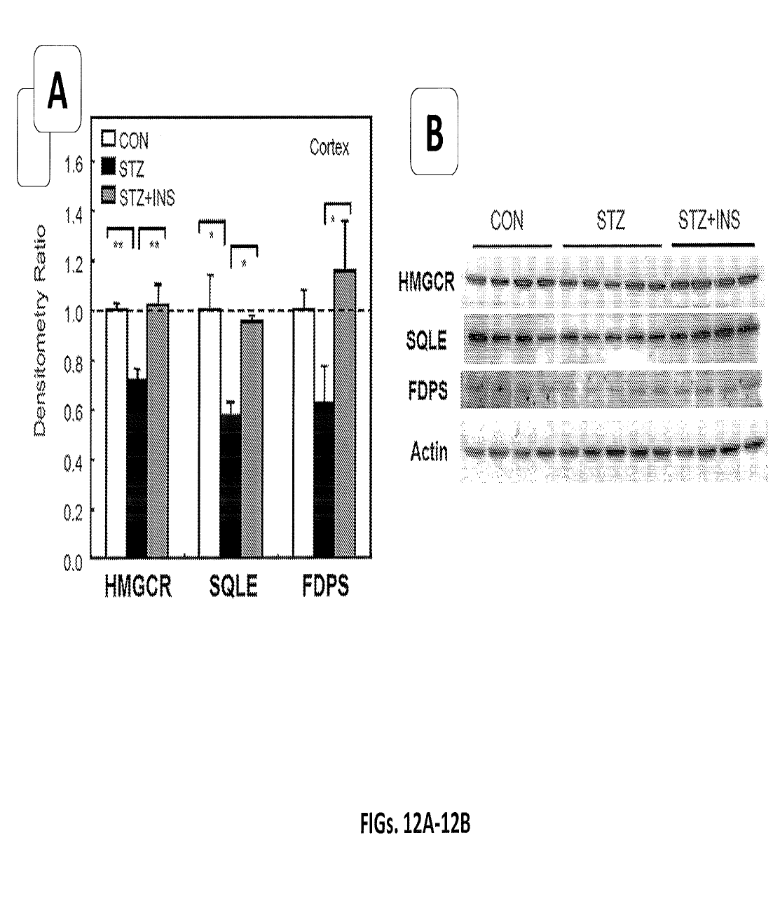

As shown in FIG. 11, down-regulation of SREBP-2 and cholesterologenic genes due to diabetes is not limited to the hypothalamus, but rather is part of a more general effect on the brain. Specifically, in the cerebral cortex, STZ-induced diabetes produced a robust reduction of these cholesterologenic genes with a 36% decrease in Srebf2 mRNA and a 38% decrease in Hmgcr mRNA. As with the hypothalamus, these were associated with parallel changes in the protein levels encoded by these transcripts in the cerebral cortex (see FIGS. 12A-12B).

### Example 3

### Role of Insulin Versus Hyperglycemia in Control of Cholesterol Synthesis

To determine whether hyperglycemia or insulin insufficiency was predominantly responsible for the above-observed suppression of cholesterol synthesis pathway in the diabetic mouse brain, we treated the STZ-induced diabetic mice with phlorizin (PHZ), a flavonoid which can decrease blood glucose levels by inhibition of renal glucose transport without restoring insulin secretion using the methods disclosed in Example 1. Additionally, phlorizin (PHZ) treatment was performed using 8-week-old C57Bl/6 mice previously treated with STZ. PHZ (Sigma) was dissolved in a solution containing 10% ethanol, 15% DMSO, and 75% saline and was injected subcutaneously (0.4 g/kg) twice daily for 10 days starting 8 days after the STZ injection. Control mice were injected with the same volume of vehicle. mRNA levels were then assessed by qPCR as disclosed in Example 1.

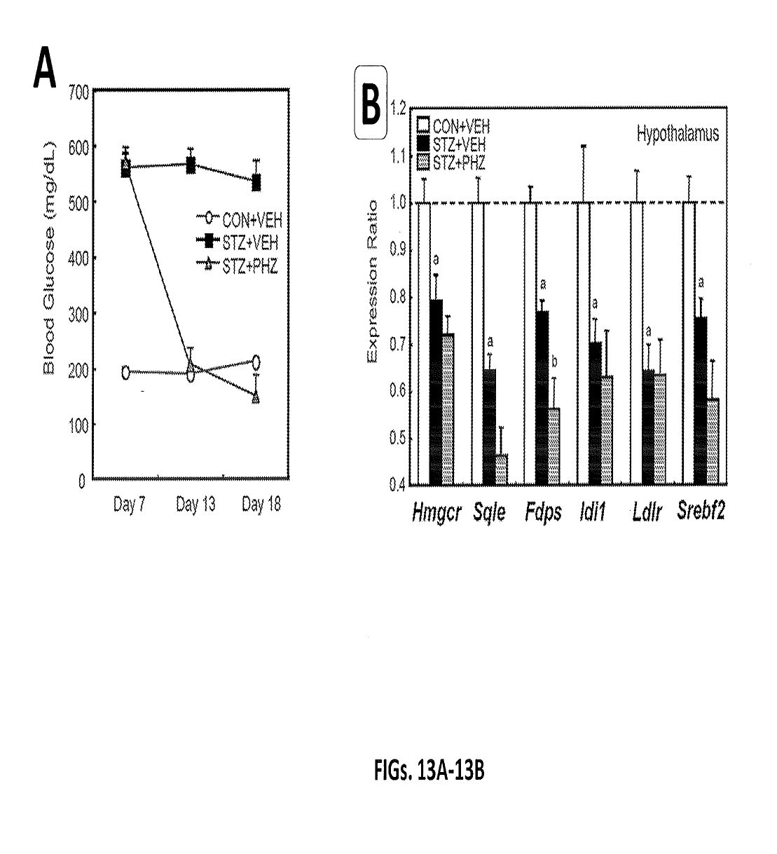

As shown in FIG. 13A, PHZ treatment efficiently normalized hyperglycemia in the STZ-diabetic mice to a level similar to that seen with insulin treatment. This reduction of blood glucose did not, however, reverse suppression of SREBP-2 and its downstream genes in the hypothalamus of diabetic mice (see FIG. 13B). This observation suggests that hyperglycemia is not the major driver for the suppression of cholesterol synthesis in diabetes.

To assess the effects of insulin, we directly administered insulin into the cerebral ventricles of STZ-induced diabetic mice by 3 injections of insulin into a catheter placed in the lateral ventricle. Briefly, seven-week-old C57Bl/6 mice were placed in a stereotactic device under anesthesia, and a 26-gauge guide cannula (Plastics One Inc., Roanoke, Va.) was inserted into the right lateral cerebral ventricle (1.0 mm posterior, 1.0 mm lateral, and 2.0 mm ventral to the bregma). A dummy stylet cannula was inserted into each cannula until used. After a 1 week recovery, mice received a single i.p. injection of STZ to induce diabetes. Twelve days later, the mice received three ICV injections of insulin (3 mU in 2 μL) or the same volume of PBS (9 AM, 7 PM and 9 AM the following day) through an internal cannula using a Hamilton microsyringe. Food intake was measured immediately before the ICV injection. Four hours after the last injection, blood glucose levels were measured, and the hypothalami collected.

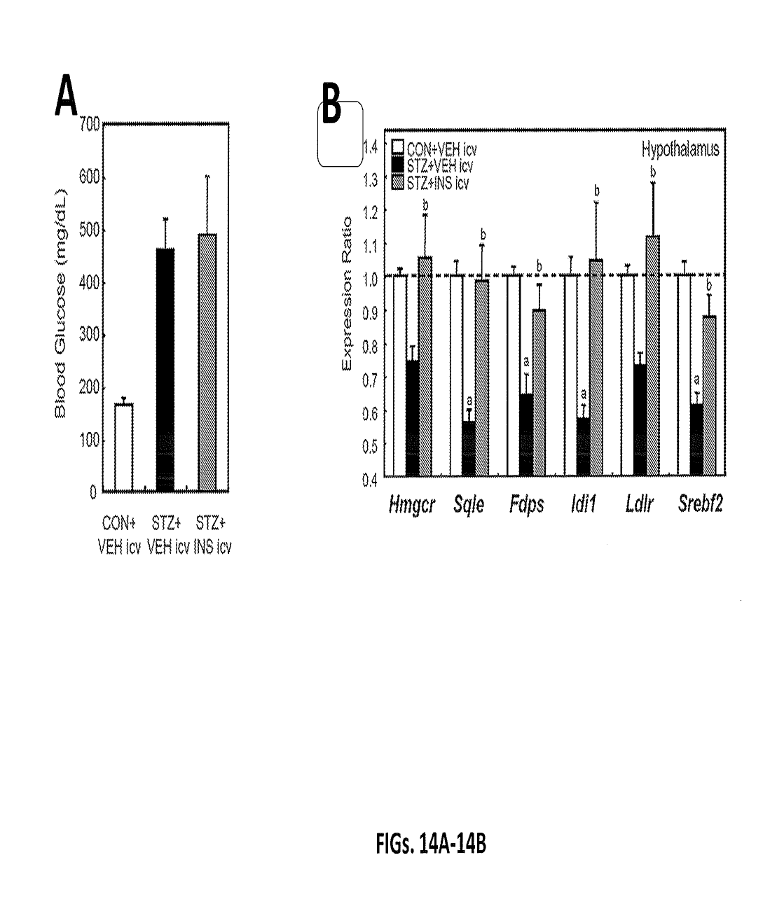

As shown in FIG. 14A. ICV injection of insulin did not affect blood glucose levels. However, as shown in FIG. 14B, suppression of SREBP-2 pathway in the hypothalamus of the diabetic mouse was almost completely normalized by insulin ICV injection. This observation supports that insulin deficiency is a major cause of the suppression.

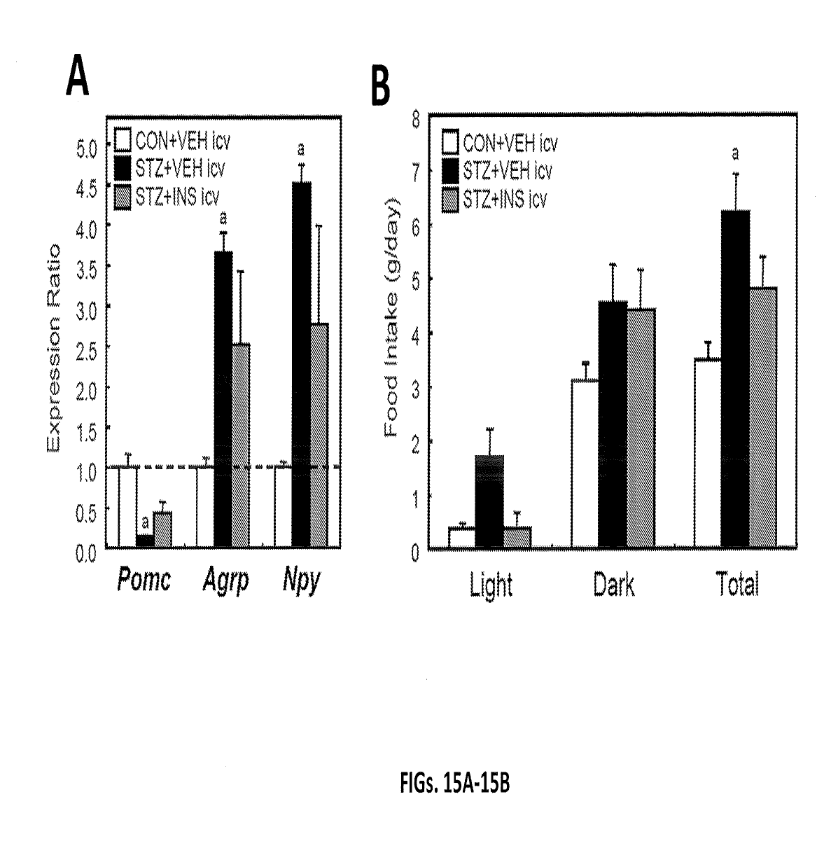

As shown in FIGS. 15A-15B, ICV injection of insulin also partially normalized the expression the neuropeptides expression involved in feeding behavior [proopiomelanocortin (Pomc), agouti-related peptide (Agrp), and neuropeptide Y (Npy)], and partly reversed the hyperphagia induced by STZ diabetes.

Together, these data indicate that insulin deficiency, not hyperglycemia, is the major driver of the altered cholesterol biosynthesis observed in the diabetic mouse.

### Example 4

### Decreased Cholesterol Synthesis in the Diabetic Brain is Accompanied by Decreased Synaptosomal Membrane Cholesterol

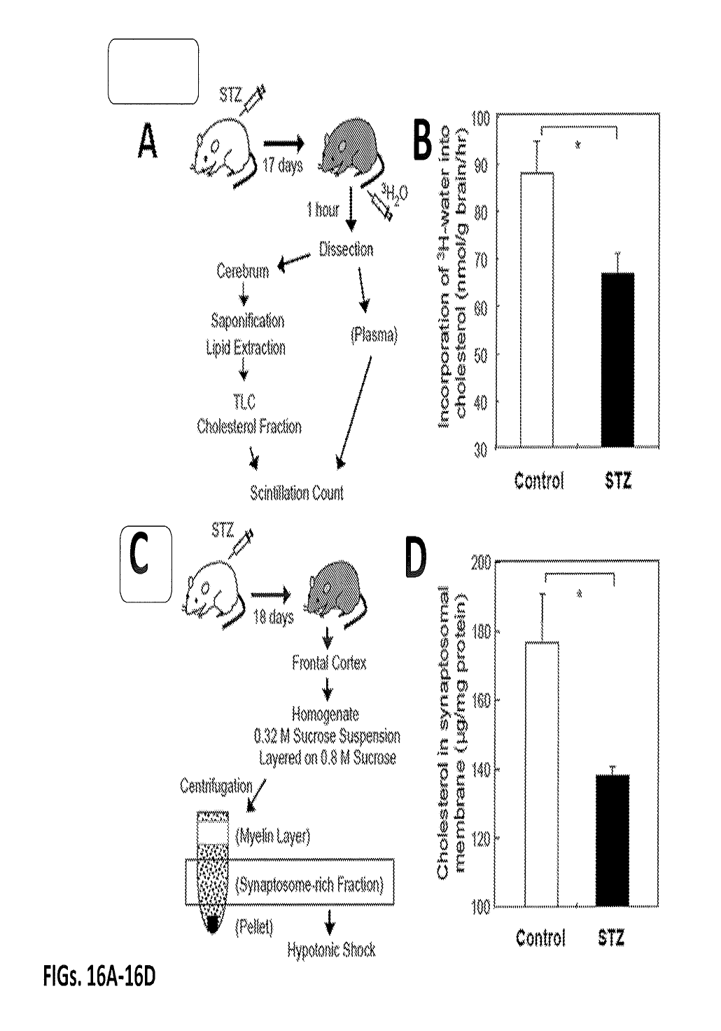

To confirm whether the above-described changes in cholesterologenic gene expression in diabetes impacts brain cholesterol synthesis, cholesterol synthesis in STZ-diabetic and control mice was assessed directly using tritiated water, as illustrated in FIG. 16A. Briefly, rates of cholesterol synthesis in the brain were measured in 7-week-old C57Bl/6 male mice 17 days after STZ or control buffer i.p. injection. Each animal was injected i.p. with 50 mCi of [3H]water in 0.2 mL of PBS. One hour later, each animal was anesthetized, blood was collected by retro-orbital puncture, and the [3H]water specific activity in the plasma was measured. The brain was removed, and the whole cerebrum was saponified with 2.5 mL of 2.5M KOH (75° C., 2 hours). Sterol-containing lipid was extracted using 10 mL hexane and 5 mL 80% ethanol. Cholesterol was isolated by thin layer chromatography (hexane:diethyl ether:glacial acetic acid=80:20:1), and the incorporated tracer was measured by scintillation counting. Synthesis rates were calculated as nmol of [3H]water incorporated into cholesterol per gram of tissue per hour.

When appropriate, synaptosomes were isolated using discontinuous sucrose density gradients centrifugation (Kolomiytseva et al., Comp. Biochem. Physiol. B. Biochem. Mol. Biol., 151:386-391 (2008)). Briefly, about 100 mg of frontal cortex tissue from 10-week-old C57Bl/6 male mice 18 days after STZ injection or human cerebral cortex tissue was homogenized in 1 mL of 0.32 M sucrose buffered with HEPES (10 mM, pH 7.4) at 4° C. by using a glass-Teflon Dounce homogenizer, then centrifuged 1,500×g for 10 min at 4° C. The supernatant (A) was collected, and the fluffy, white layer above the pellet was discarded. The pellet obtained after centrifugation was resuspended in 1 mL of 0.32 M sucrose buffer and centrifuged again. The supernatants obtained (B) was combined with supernatant (A) and centrifuged again at 9,000×g for 20 min, 4° C. The pellet (crude synaptosomal fraction containing myelin, synaptosomes and mitochondria) was resuspended in 500 μL of 0.32 M sucrose buffer, and every 250 μL of the suspension was layered over 1 mL of 0.8 M sucrose buffer. After centrifugation at 9,000×g for 25 min, pellets were resolved into three fractions: a thick white band at the 0.32-0.8 M sucrose interface (mainly myelin); pellets dispersed in the 0.8 M sucrose solution (mainly synaptosomes); and a pellet (mainly mitochondria). The synaptosome-rich fraction in the 0.8 M sucrose buffer was diluted with 0.1 M sucrose to obtain a final 0.32 M sucrose buffer, and centrifuged under the same conditions as the previous gradient. The pellets of synaptosomes was washed by ice-cold PBS and resuspended in 500 μL of 5 μM Tris-HCL buffer (pH 7.4), then placed on ice for 30 min. The pellet containing synaptosomal membranes was lysed with buffer containing 1% Triton-X. Cholesterol was measured by an enzymatic assay (Wako Chemicals). Protein concentrations were measured by BCA assay.

As shown in FIG. 16B, in vivo cholesterol synthesis in the brain was reduced by 24% (P<0.05) in diabetic mice, closely paralleling the decrease in cholesterologenic enzymes reported above via qPCR and immunoblotting. Since much of brain cholesterol is in slowing turning over myelin (Dietschy and Turley, J. Lipid. Res., 45:1375-1397 (2004)), it is perhaps not surprising that no change was observed in total cholesterol content in the cerebral cortex after only 18 days of diabetes (13.7±0.4 versus 14.1±0.4 mg/g of tissue: P=0.49). However, as shown in FIGS. 16C-16D, the reduced cholesterol synthesis was reflected with a significant and parallel 22% decrease in cholesterol content of isolated synaptosomal membranes from the brains of diabetic mice.

### Example 5

### Insulin Regulates Cholesterol Synthetic Processes in Humans

To confirm that cholesterol synthetic processes are regulated by insulin in humans as is described herein for mice, cholesterol synthetic gene expression patterns and synaptosomal cholesterol content were assessed in cerebral cortices from 16 elderly (75-99 years old) humans.

Autopsies were performed as a part of an ongoing, prospective, longitudinal, population-based study of aging and cognitive decline (Adult Changes in Thought Study) (Sonnen et al, 2009). The middle frontal gyrus and superior and middle temporal gyri were flash frozen in liquid nitrogen and stored at −80° C. Tissues from 7 males and 9 females were analyzed; the age ranged from 81 to 99 years for males and from 75 to 96 years for females. All the autopsies were performed within 7 hours of death. Samples were assessed using methods disclosed in the above Examples.

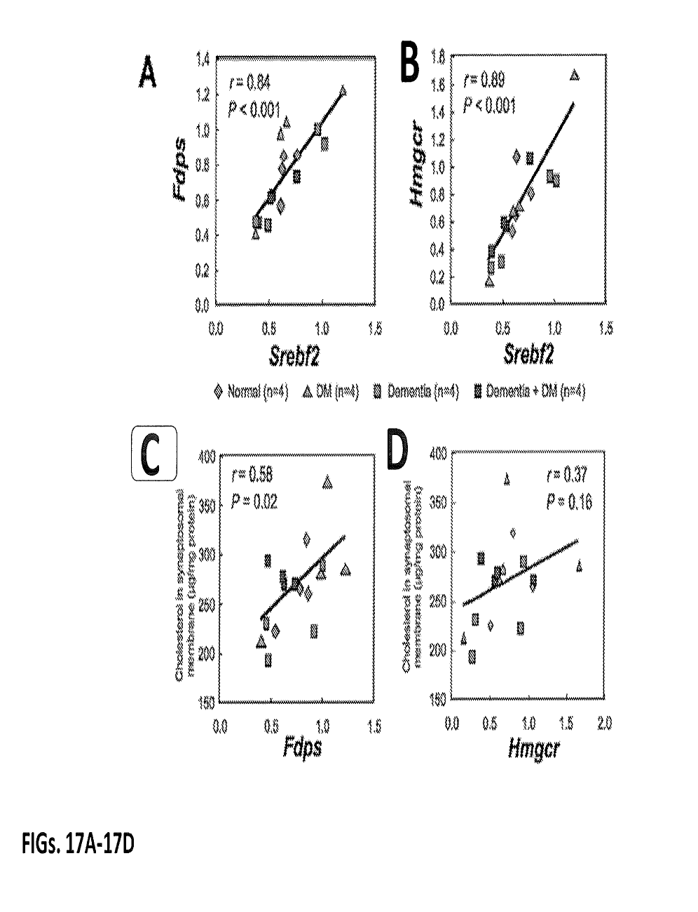

As shown in FIGS. 17A-17B, in the cortices there was a strong positive correlation between the levels of Srebf2 mRNA and those for two of its downstream targets—Hmgcr and Fdps. There was also a significant positive correlation (r=0.58, P=0.02) between cholesterol content in isolated synaptosomal membranes and the mRNA expression of Fdps, (see FIGS. 17C-17D), suggesting that factors which control expression of genes for cholesterol synthesis may also affect synaptosomal cholesterol in human brains.

### Example 6

### Diabetes Suppresses Synthesis of Sterols Other than Cholesterol in the Brain

To analyze whether levels of sterols other than cholesterol, including cholesterol precursors, were reduced in diabetes, levels of cholesterol precursors and derivatives were measured in the brains from 10-week-old C57Bl/6 mice, 18 days after STZ or control buffer i.p. injection. Lipids were extracted from the sagitally-sectioned half brains. Sterols were resolved by HPLC, identified by QTRAP mass spectrometer (Applied Biosystems), and quantified by comparison of the areas under the elution curves derived from the detection of endogenous compounds and isotopically labeled standards (McDonald et al., Methods Enzymol., 432:145-170 (2007)).

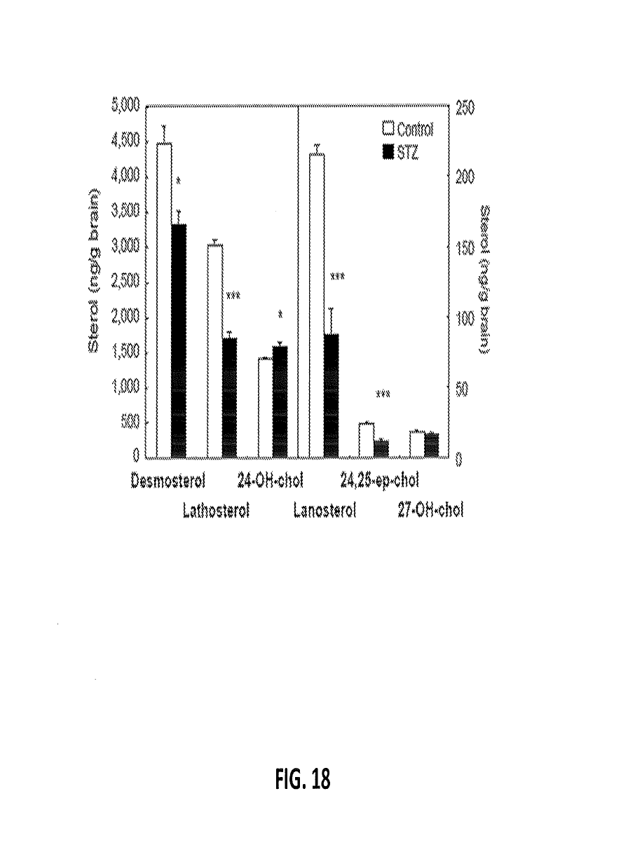

As shown in FIG. 18, broad suppression of the cholesterologenic pathway in diabetes affects multiple steps of cholesterol biosynthesis in the brain. Specifically, cholesterol precursors, such as desmosterol, lathosterol, and lanosterol, were reduced by 26%, 44%, and 60% respectively, in the brain of STZ-induced diabetic mice. Thus in addition to its effects on cholesterol synthesis, diabetes may suppress synthesis of other related lipid products, including isoprenoids, dolichols and ubiquinone in the brain.

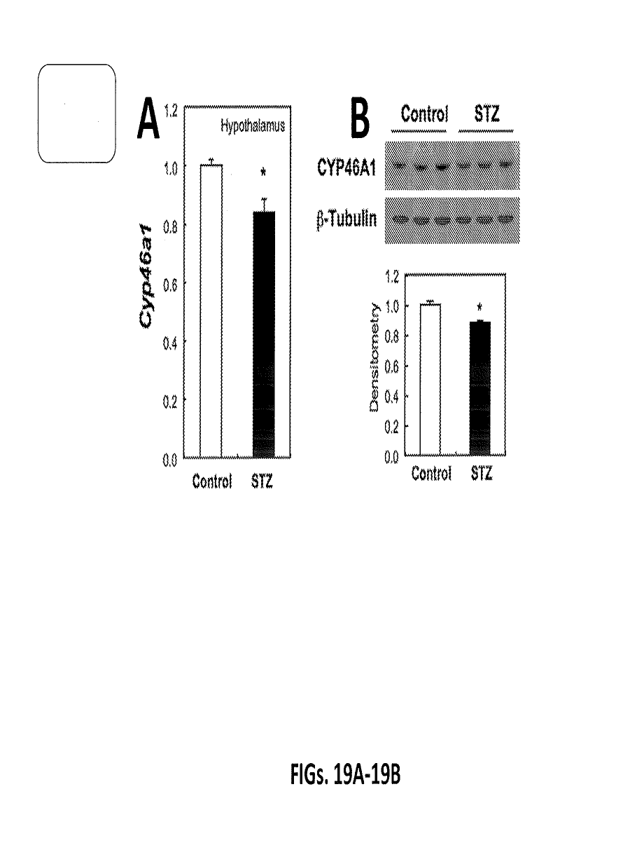

Cholesterol in the brain is reportedly converted to 24-hydroxycholesterol, which can spontaneously diffuse into the circulation, by a neuronal enzyme cholesterol 24-hydroxylase (CYP46A1) (Russell et al., Annu. Rev. Biochem., 78:1017-1040 (2009)). As shown herein, although the expression of CYP46A1 was mildly down-regulated (see FIG. 19), the content of 24-hydroxycholesterol in the STZ-diabetic brain was slightly increased (12%) (FIGS. 19A-19B). Further, content of other oxysterols, such as 24,25-epoxycholesterol and 27-hydroxycholesterol, on the other hand, were decreased (FIG. 18).

### Example 7

### Insulin Activates Cholesterol Biosynthesis Pathway in Cultured Neurons and Glial Cells

To dissect the cell type and factors regulating the changes in cholesterol biosynthesis, neuron and glial cells (astrocytes) were isolated and cultured from the cortices of 16-day-old C57Bl/6 mouse embryos and 1-day-old neonates, respectively. Briefly, the cerebral cortex was dissected from 0-1 day old for glia and day 16 embryonic C57Bl/6 mice under aseptic conditions. Large blood vessels were carefully removed under the microscope. The tissue was coarsely minced by forceps in ice-cold L15 medium and rinsed in ice-cold PBS for 5 times. The cortex was digested in 0.25% trypsin and 10 μg/ml DNase (Roche) at 37° C. for 15 minutes. After adding an equal volume of horse serum, the tissue was centrifuged at 600×g for 5 minutes. The pellet was suspended in Minimum Essential Medium (MEM) containing 10% horse serum and filtered through a 40 μm nylon cell strainer (BD Falcon). The cell suspension was plated on poly-L-lysine (PLL)-coated 12 well plates in MEM with 10% horse serum. Medium was replaced every 4 days after the initial plating.

Primary culture cortical neurons were prepared from E16 embryonic C57Bl/6 mouse brains. Neurons were plated at a density of 2×105 cells/cm2 on PLL-coated plates in Dulbecco's modified Eagle medium (DMEM) and Ham's F-12 medium (1:1) supplemented with 5% fetal bovine serum and 5% horse serum. Cytosine arabinoside (10 μM) was added to the culture medium 2 days after plating. The medium was replenished by half every 4 days.

For insulin stimulation, cells were serum deprived and pre-incubated with medium containing 0.5% bovine serum albumin (BSA) for 6 hours, then stimulated with 0 to 100 nM insulin for 6 hours. For glucose challenge, cells were incubated in medium containing serum and 5 mM or 25 mM glucose for 72 hours. Cells were harvested between 18 and 22 days after the initial plating.

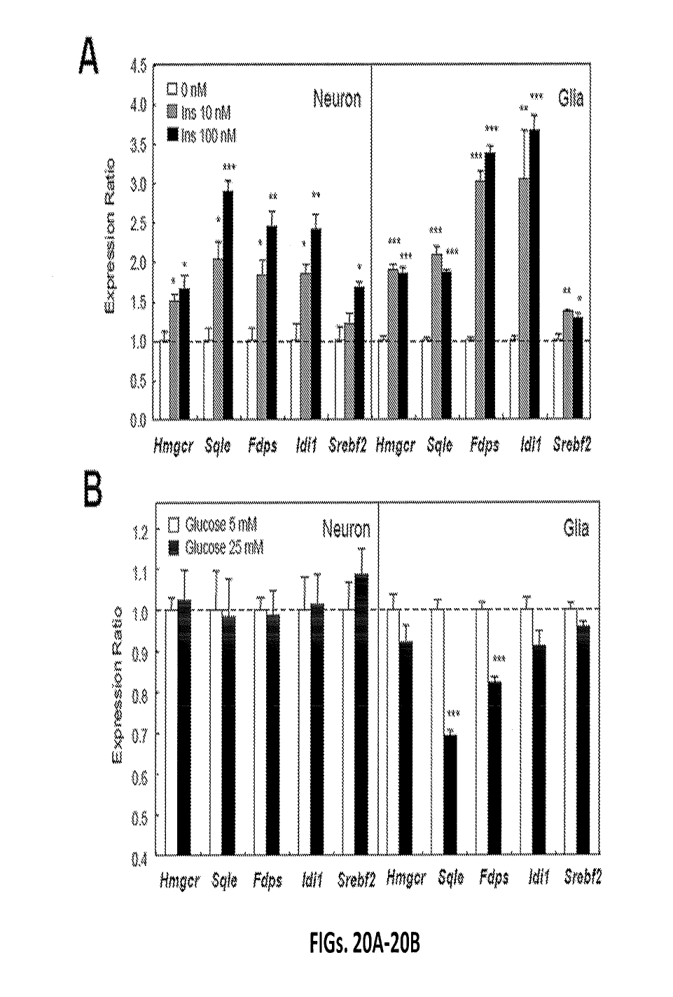

As shown in FIG. 20A, in both astrocytes and glial cells, 6 hours of exposure to insulin (10-100 nM) stimulated expression of all of the cholesterologenic genes by 30 to 270%. Furthermore, incubation of cells with high concentrations (25 mM) of glucose for 3 days suppressed mRNA of some cholesterologenic genes, such as Sqle and Fdps, in glial cells, but had no effect on any of the cholesterologenic genes in cortical neurons (see FIG. 20B), consistent with the in vivo studies (see FIGS. 13-15). These observations suggest that insulin, not hyperglycemia, is responsible for the altered expression of genes involved in cholesterol synthesis in brain in diabetes.

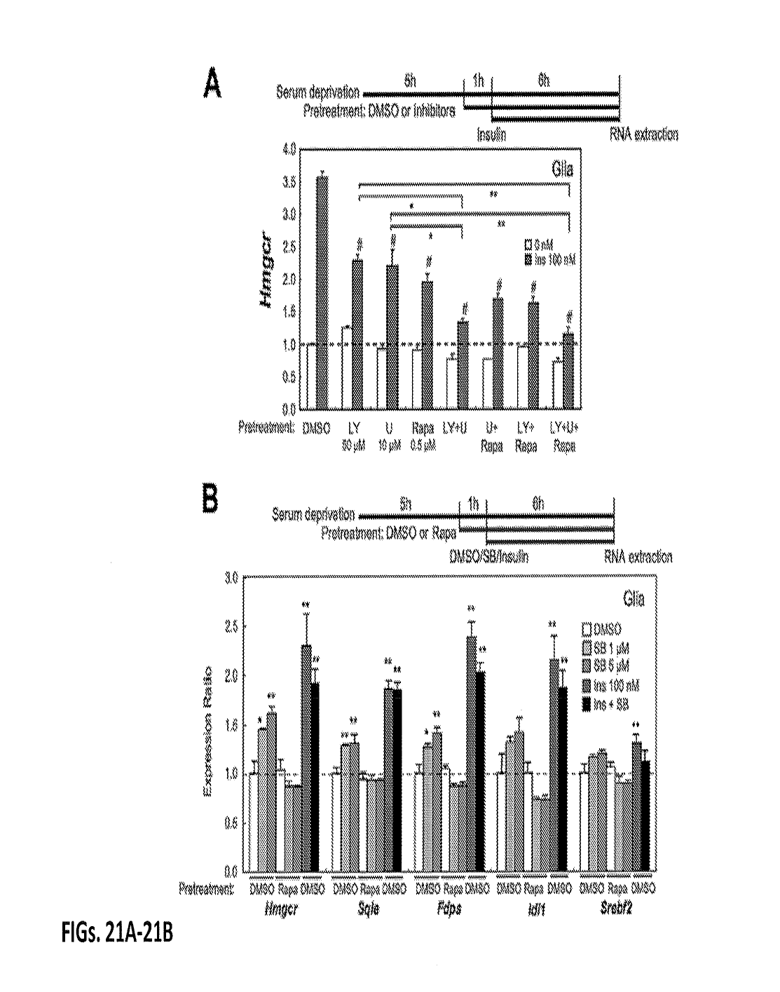

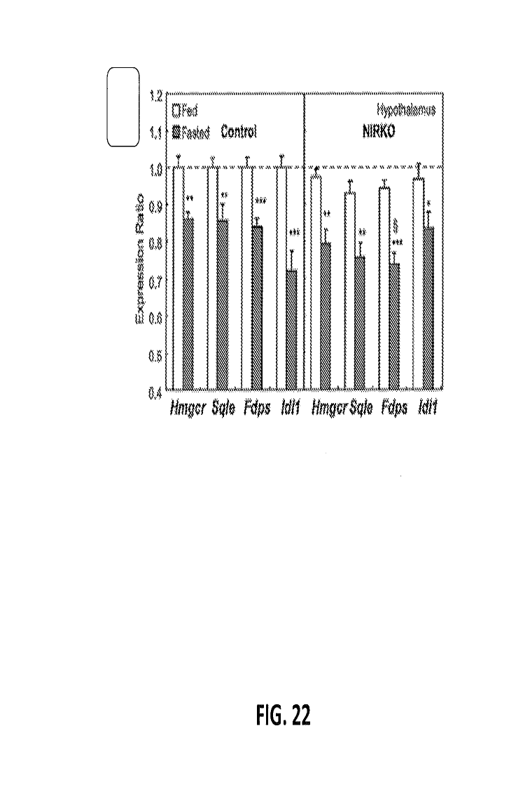

Effects of insulin on cholesterol synthetic genes involve multiple signaling pathways. In liver, SREBP-effects on lipogenesis involve Akt, atypical protein kinase C, and mTORC1 activity (Li et al., Proc. Natl. Acad. Sci., USA, 107:3441-3446 (2010); Porstmann et al., Cell. Metab., 8:224-236 (2008); Taniguchi et al., Cell. Metab., 3:343-353 (2006)). As shown in FIG. 21A-21B, in cultured neurons, rapamycin, as well as the PI 3-kinase inhibitor LY294002 and the MEK inhibitor U0126, partially suppressed insulin's effect on Hmgcr induction. Further, the observed effects were additive suggesting that the mTORC1, PI 3-kinase and MAP-kinase pathways act together in Hmgcr induction by insulin (FIG. 21A). Treatment of cultured neurons with the glycogen synthase kinase (GSK) 3 inhibitor SB216763 mimicked insulin's effects on this pathway producing a broad increase in expression of cholesterol synthetic genes that was also inhibited by pretreatment of cells with the mTORC1 inhibitor rapamycin (FIG. 21B). Since the mTORC1 pathway is activated by nutrients (Avruch et al., 2009), as well as hormones like insulin, this pathway may play a role in the decrease of cholesterol synthetic genes induced by fasting (see FIG. 22).

### Example 7

### Loss of SREBP-2 Reduces Pre- and Post-Synaptic Markers in Hippocampus Neurons

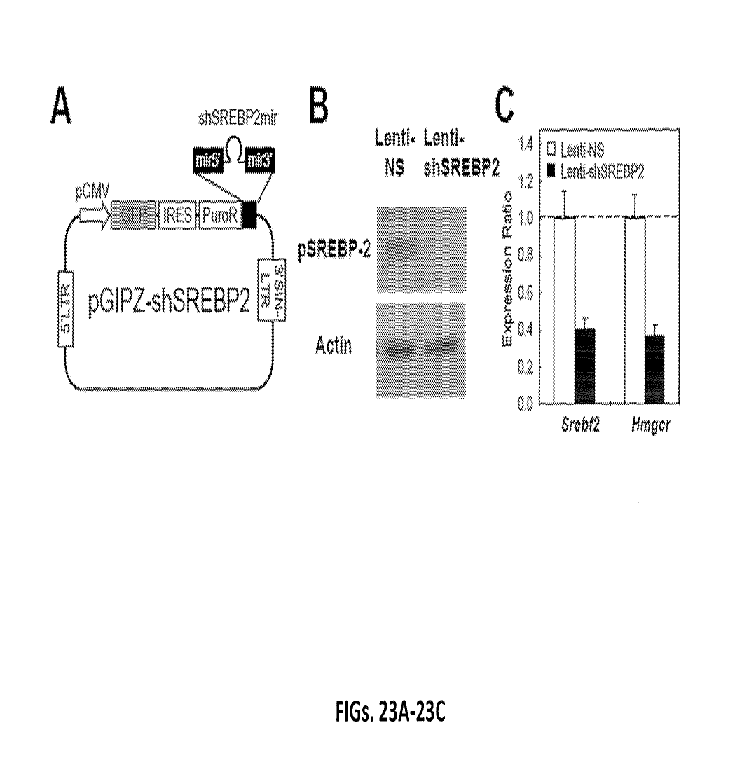

Cholesterol is crucial for synaptic structure, function, and genesis (Mauch et al., Science, 294:1354-1357 (2001)). To investigate the effects of reduced cholesterologenic genes in brain, a SREBP-2 silencing lentivirus vector (Lenti-shSREBP2), which co-expressed Srebf2 shRNA with green fluorescent protein (GFP) under the control of cytomegalovirus promoter was constructed (see FIG. 23A). Lentiviral vector plasmids for murine SREBP-2 shRNA (GIPZ Lentiviral shRNAmir, Open Biosystems, Huntsville, Ala.) and control non-silencing (NS) shRNA were packaged by co-transfection with packaging plasmids in HEK293T cells (Trans-Lentiviral Packaging System, Open Biosystems). Viral particles were concentrated by ultracentrifugation. For confocal assays, primary cultures of hippocampus were prepared from 0-1 day old C57Bl/6 mice using a Papain Dissociation System (Worthington Biomedical Corporation, Lakewood, N.J.). Dissociated cells were plated on poly-L-lysine-coated cover glasses (Carolina Biological Supply, Burlington, N.C.) at 6×104 cells/cm2 and cultured in Neurobasal medium (Invitrogen) containing the 1×B27 supplement (Invitrogen), 125 μM GlutaMax (Invitrogen) and 25 μM glutamic acid. Neurons were infected with lentivirus vectors after 2 days in vitro (DIV). On 4 DIV the culture medium was changed to Neurobasal medium supplemented with 1×B27, 125 μM GlutaMax and 10 μM cytosine arabinoside (Sigma). For immunostaining, hippocampal neurons were fixed with 4% PFA for 15 min, and permeabilized with 0.25% Triton X-100 in PBS for 5 min. After cells were blocked with PBS containing 10% BSA for 30 min, they were incubated with the first antibody (anti-PSD95, Abcam, 1:500; anti-VAMP2, Abcam, 1:1000; anti-MAP2, Millipore, 1:200) in PBS containing 3% BSA overnight at 4° C., washed with PBS, and incubated with the second antibody in PBS containing 3% BSA for 1 hour. After the samples were washed with PBS, they were embedded in fluorescence mounting medium (DAKO). The images were obtained by confocal microscopy (Zeiss LSM-410), and quantified by ImageJ software (NIH).

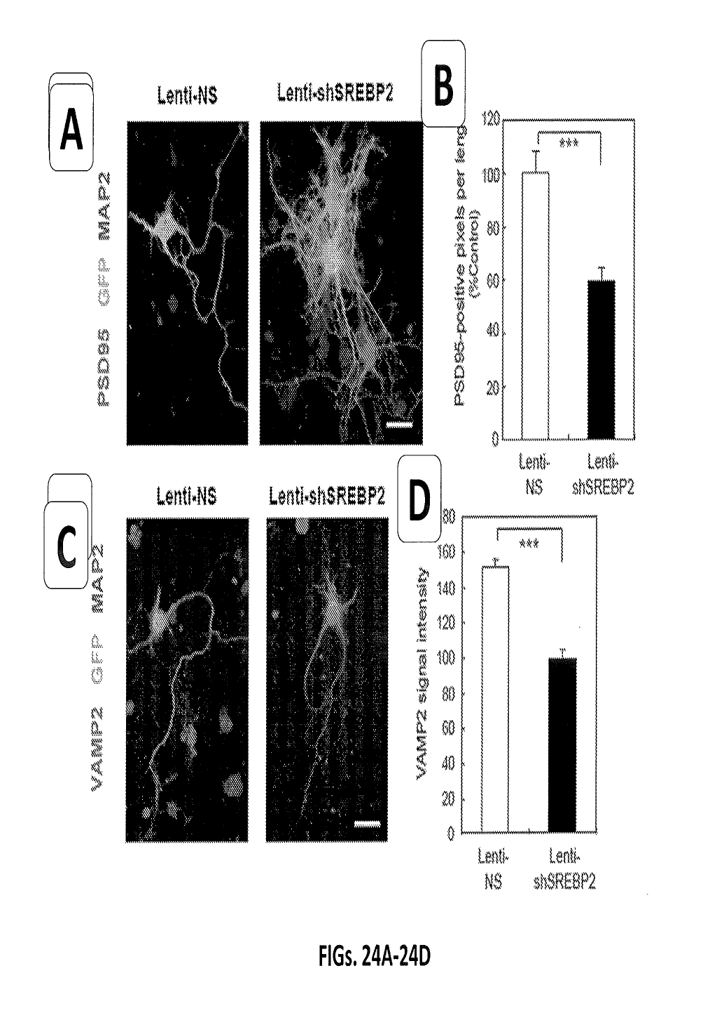

As shown in FIG. 23B, Western blot analysis indicated that infection with Lenti-shSREBP2 vector suppressed SREBP-2 expression by 90% in N25/2 mouse hypothalamic neuronal cells in culture compared with a control non-silencing vector. Further, as shown in FIG. 23C, transient infection with the Lenti-shSREBP2 without selection caused 60% reduction of Srebf2 and Hmgcr genes in primary cultured mouse hippocampal neurons (FIG. 6C). These knock-downs resulted in a 40% reduction (P<0.001) in density of synapse formation on the neurites as indicated by staining with the post-synaptic marker PSD95 (see FIGS. 24A-24B). In the SREBP2 knockdown neurites, there was also a 34% decrease in the staining intensity of the synaptic vesicle marker VAMP2 (see FIG. 24C-24D), consistent with the role of cholesterol in synaptic vesicle biogenesis (Thiele et al., Nat. Cell. Biol., 2:42-49 (2000)) and exocytosis (Lang et al., EMBO J., 20:2202-2213 2001)).

These findings provide a link between diabetes and altered synapse function.

### Example 8

### Knockdown in the Hypothalamus Affects Feeding Behavior and Endocrine Homeostasis

To elucidate the physiological role of SREBP-2 in the brain in vivo, the Lenti-shSREBP2 vector was injected directly into the hypothalami of C57Bl/6 mice. 7 to 8 week-old male C57Bl/6 mice were used. The mice were housed in individual cages, and given diet pellets containing 10% fat by kilocalories (D12450B, Research Diet Inc.) and free access to water prior to the experiments. Mice were placed in a stereotactic device under anesthesia, and a 33-gauge cannula (Plastics One Inc.) was inserted into the hypothalamus (0.5 mm posterior, ±0.5 mm lateral, and 5.7 mm ventral from the bregma). Lentivirus vector (˜108 TU/mL, 0.5 μL) was injected into the ventral hypothalamus bilaterally between 9-10 AM. Body weight of all mice was monitored daily, and food intake was measured twice a day at 9-10 AM and 6-7 PM, from 15 days after the injection for consecutive 12 days. Plasma norepinephrine levels were measured using an ELISA kit (Immuno-Biological Laboratories Inc., Minneapolis, Minn.). CLAMS studies were performed over a three day period following one day of acclimation. Insulin tolerance test was performed by intraperitoneal injection of 1 U/kg BW insulin (NovoLog, Novo Nordisk Inc.). Glucose tolerance tests were performed by intraperitoneal injection of 2 g dextrose/kg BW after an overnight fast.

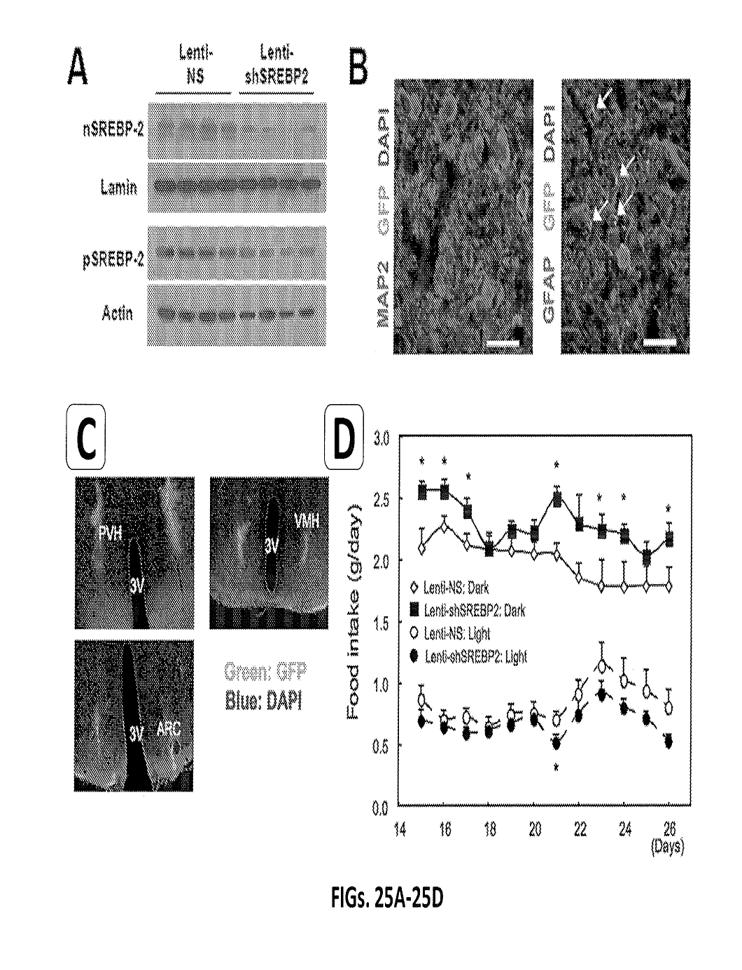

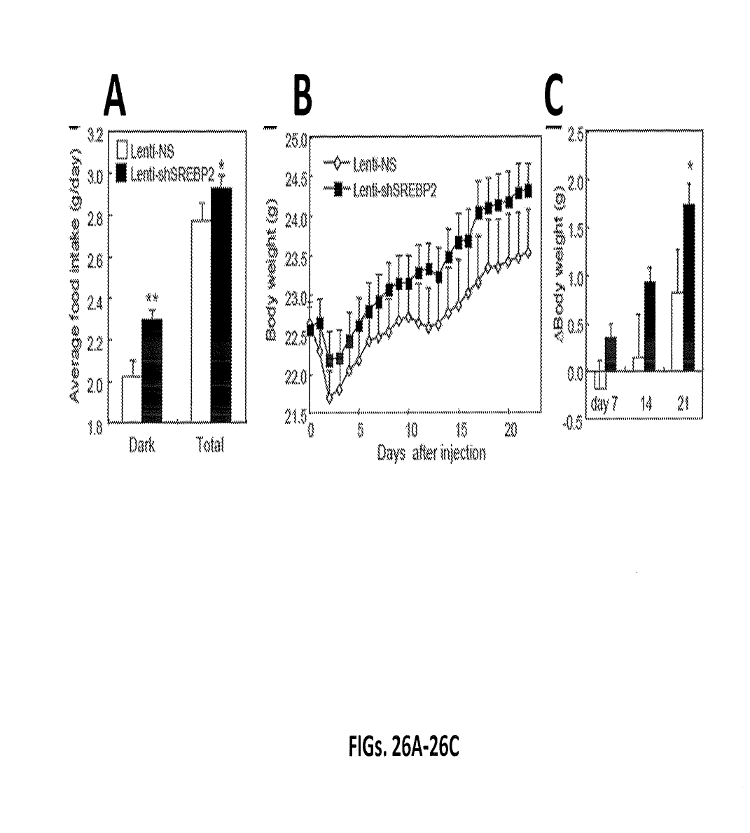

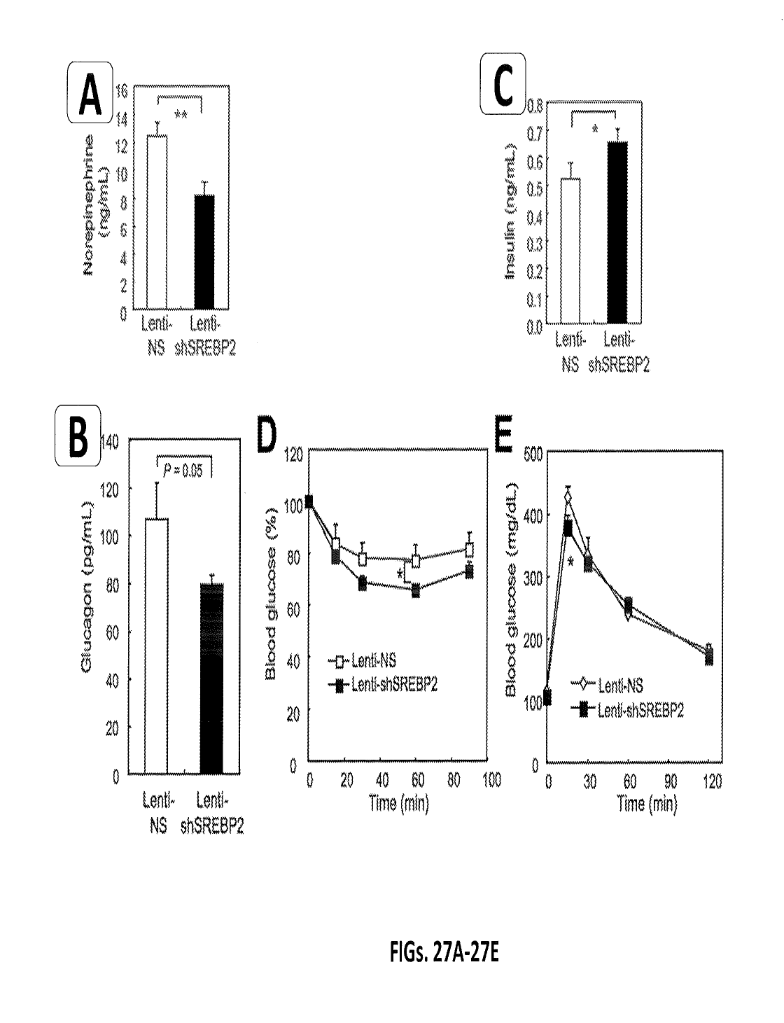

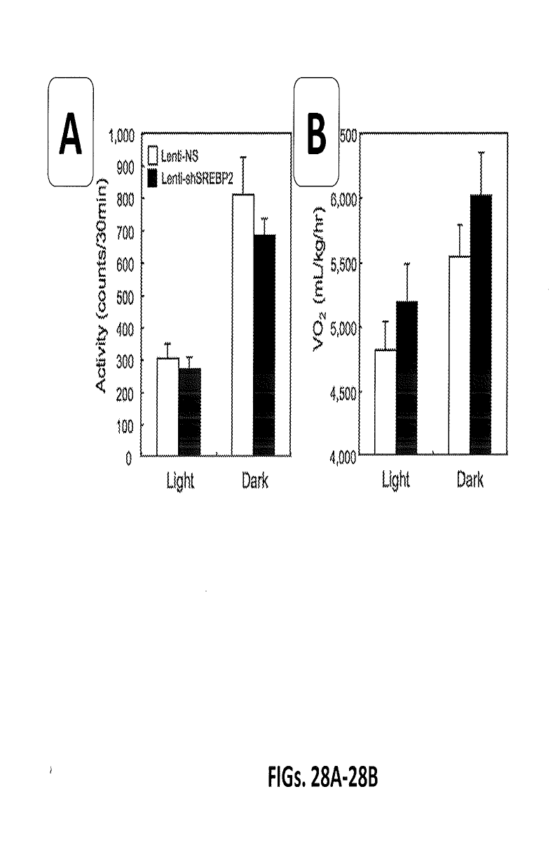

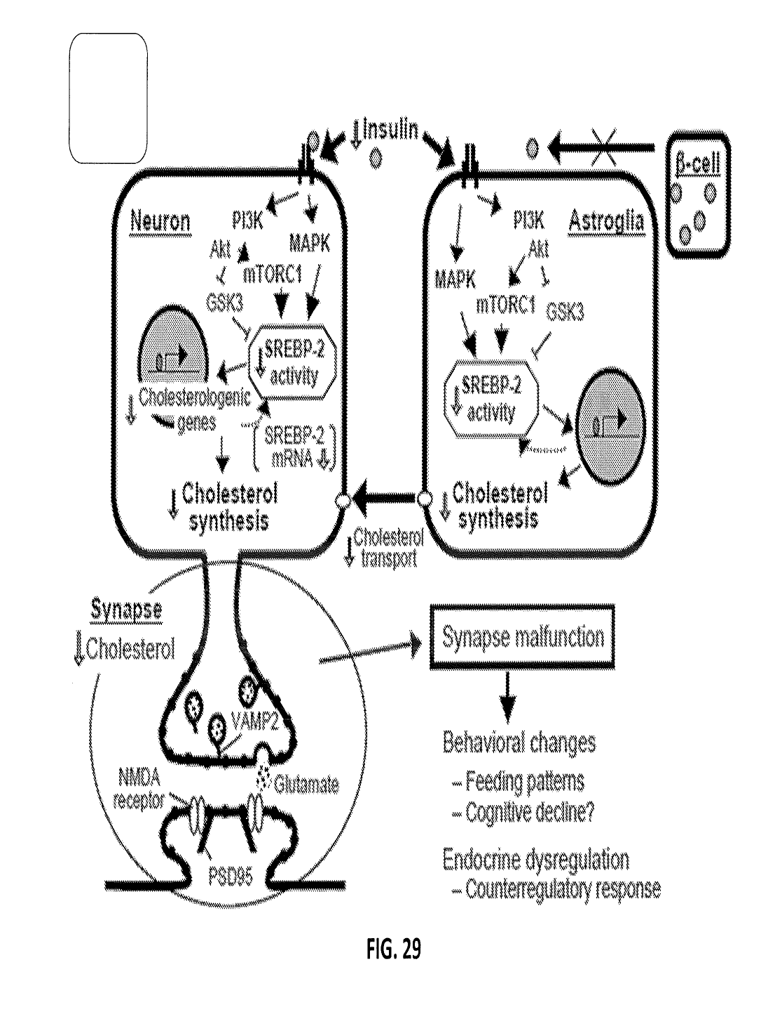

As shown in FIG. 25A., intrahypothalamic Injection of Lentivirus Vectors produced a 33% reduction in the precursor and 44% reduction in the nuclear forms of SREBP-2 in the ventral hypothalamus. GFP fluorescence, an indicator of lentivirus infection, was observed in neuronal cell bodies and some astrocyte processes (FIG. 25B) in the paraventricular hypothalamus (PVH), ventromedial hypothalamus (VMH), and arcuate nucleus (ARC) (FIG. 25C). After a brief initial decline in food intake in all mice receiving intra-hypothalamic injection, mice with knockdown of SREBP-2 exhibited a 14% increase in nocturnal food intake (P<0.01) and a similar increase in total daily food intake (FIG. 26A and FIG. 25D). As a result, body weight gain over the 21 days following injection was significantly greater in the mice treated with Lenti-shSREBP2 compared to controls (FIGS. 26B and 26C). Neurons in the VMH are largely glutamatergic (Tong et al., Cell. Metab., 5:383-393 (2007)) and have been shown to mediate counter-regulatory responses to hypoglycemia (Borg et al., J. Clin. Invest., 93:1677-1682 (1994)). In this regard, mice with hypothalamic knockdown of SREBP-2 exhibited a 34% reduction of circulating norepinephrine (see FIG. 27A) and glucagon (see FIG. 27B) in the fasted state and a small increase in fasting insulin levels (see FIG. 27C). Insulin tolerance tests (FIG. 27D) and glucose tolerance testing (FIG. 27E) showed either no change or a very modest improvement, and there was no change in physical activity or whole body oxygen consumption rate as assessed in metabolic cages (FIGS. 28A-28B).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### INCORPORATION BY REFERENCE

The disclosure of each and every US and foreign patent and pending patent application and publication referred to herein is specifically incorporated by reference herein in its entirety.

What is claimed is:

1-37. (canceled)

38. A method of treating a neuropathy or neuronal dysfunction that is associated with reduced cholesterol levels in a subject, the method comprising:
selecting a subject having a neuropathy or neuronal dysfunction that is associated with reduced cholesterol levels; and
administering to the subject a therapeutically effective amount of an agent selected from the group consisting of insulin and insulin analogues that cross the Blood Brain Barrier (BBB).

39. The method of claim 1, wherein the neuropathy is diabetic neuropathy.

40. The method of claim 2, wherein the agent is administered via inhalation.

41. The method of claim 3, wherein the subject has type 1 diabetes.

42. The method of claim 3, wherein the subject has type 2 diabetes.

43. The method of claim 1, further comprising assessing the neuropathy or neuronal dysfunction.

44. The method of claim 1, wherein the subject does not have diabetes.

45. The method of claim 1, wherein the neuropathy is Alzheimer's disease.

46. The method of claim 1, wherein the neuropathy is a cognitive disorder.

47. The method of claim 9, wherein the agent is administered via inhalation.

48. The method of claim 1, wherein the agent is the insulin analogue Glargine, Exu-bera®, Ora-Lyn®, or an analogue of either one.

49. The method of claim 1, wherein the subject has not been administered insulin or an insulin analog prior to its administration for treating a neuropathy or neuronal dysfunction.

50. The method of claim 1, wherein the insulin or insulin analog is being administered with the sole purpose of treating the neuropathy or neuronal dysfunction.

51. The method of claim 1, wherein the subject was being administered insulin or an insulin analog prior to administration of insulin or an insulin analog for treating the neuropathy or neuronal dysfunction, and wherein the dose of insulin or insulin analog for treating the neuropathy or neuronal dysfunction is different from the dose of insulin or insulin analog that the subject received prior to administration of insulin or insulin analog for treating the neuropathy or neuronal dysfunction, and the dose of insulin or insulin analog is changed to the dose of insulin or insulin analog that is administered for treating the neuropathy or neuronal dysfunction.

52. The method of claim 14, wherein the dose of insulin or insulin analog for treating the neuropathy or neuronal dysfunction is higher than the dose of insulin or insulin analog that was administered to the subject prior to administration of insulin or insulin analog for treating the neuropathy or neuronal dysfunction, and the subject is being administered the higher dose of insulin or insulin analog for treating the neuropathy or neuronal dysfunction.

53. The method of claim 1, wherein the subject was being administered insulin or an insulin analog prior to administration of insulin or an insulin analog for treating the neuropathy or neuronal dysfunction, and wherein the regimen of insulin or insulin analog administration for treating the neuropathy or neuronal dysfunction is different from the regimen of administration of insulin or insulin analog that the subject received prior to administration of insulin or an insulin analog for treating the neuropathy or neuronal dysfunction, and the regimen of administration of insulin or insulin analog is changed to that for treating the neuropathy or neuronal dysfunction.

54. The method of claim 16, wherein the insulin or insulin analog is administered more frequently for the treatment of neuropathy or neuronal dysfunction than administration of insulin or insulin analog prior to administration of insulin or insulin analog for treating the neuropathy or neuronal dysfunction.

55. The method of claim 17, wherein the insulin or insulin analog is administered more frequently and at a higher dose than the insulin or insulin analog was administered to the subject prior to the start of the administration of insulin or insulin analog for the treatment of the neuropathy or neuronal dysfunction.

56. The method of claim 1, wherein the subject was being administered insulin or an insulin analog prior to administration of insulin or an insulin analog for treating the neuropathy or neuronal dysfunction, and wherein the insulin or insulin analog that is being administered for treating the neuropathy or neuronal dysfunction is different from the insulin or insulin analog that the subject received prior to administration of insulin or an insulin analog for treating the neuropathy or neuronal dysfunction, and the insulin or analog is changed to that for treating the neuropathy or neuronal dysfunction.

57. The method of claim 19, wherein the insulin or insulin analog that was being administered prior to administration of insulin or insulin analog for treating a neuropathy or neuronal dysfunction was not a form of insulin or analog of insulin with effective crossing of the BBB, and wherein the insulin or insulin analog that is being administered for treating the neuropathy or neuronal dysfunction is a form of insulin or insulin analog that more effectively crosses the BBB relative to the insulin or insulin analog that was administered to the subject prior to administration of insulin or insulin analog for treating the neuropathy or neuronal dysfunction.

58. The method of claim 20, wherein the insulin or insulin analog that was being administered prior to administration of insulin or an insulin analog for treating a neuropathy or neuronal dysfunction was not an inhalable form of insulin or insulin analog, and wherein the insulin or insulin analog that is being administered for treating a neuropathy or neuronal dysfunction is an inhalable form of insulin or insulin analog.

59. The method of claim 1, wherein the subject was being administered insulin or an insulin analog prior to administration of insulin or insulin analog for treating the neuropathy or neuronal dysfunction, and wherein the insulin or insulin analog that is being administered for treating the neuropathy or neuronal dysfunction is different from the insulin or insulin analog that the subject received prior to administration of insulin or an insulin analog for treating the neuropathy or neuronal dysfunction, and the subject is being administered both (i) the insulin or insulin analog that was administered prior to administration of insulin or insulin analog for treating the neuropathy or neuronal dysfunction and (ii) the insulin or analog for treating the neuropathy or neuronal dysfunction.

60. The method of claim 22, wherein the subject is being administered a form of insulin or insulin analog that does not effectively cross the BBB prior to and during administration of insulin or insulin analog for treating a neuropathy or neuronal dysfunction, and the subject is further being administered a form of insulin or insulin analog that crosses the BBB more effectively than the form of insulin or insulin analog that was being administered to the subject prior to administration of insulin or insulin analog for the treatment of the neuropathy or neuronal dysfunction.

61. The method of claim 22, wherein the subject is being administered a noninhalable form of insulin or insulin analog prior to and during administration of insulin or insulin analog for treating the neuropathy or neuronal dysfunction, and the subject is further being administered an inhalable form of insulin or insulin analog for the treatment of the neuropathy or neuronal dysfunction.

62. The method of claim 1, wherein said insulin or insulin analog increases SREBP-2 expression or activity in the CNS of the subject.

63. A method for increasing cholesterol synthesis in the central nervous system (CNS) of a subject, the method comprising:
selecting a subject in need of increased cholesterol synthesis in the CNS; and
administering to the subject a therapeutically effective amount of an agent selected from the group consisting of insulin and insulin analogues that cross the BBB.

64. The method of claim 26, wherein said insulin or insulin analog increases SREBP-2 expression or activity in the CNS of the subject.

65. A method for identifying a candidate compound that increases cholesterol synthesis in the CNS, the method comprising:
contacting a cell comprising SREBP-2 with a compound; and
detecting the level of SREBP-2 in the cell or a sample therefrom, wherein an increase in the level of SREBP-2 following contacting the cell with the compound indicates that the compound is a candidate compound that increases cholesterol synthesis in the CNS.

66. The method of claim 28, further comprising comparing the level of SREBP-2 in the cell following contacting the cell with the compound with a control level of SREBP-2 in the cell prior to contacting the cell with the compound.

67. The method of claim 29, wherein the cell comprises a genetic reporter that is transcriptionally regulated by SREBP-2.

68. The method of claim 30, further comprising administering the candidate compound to an animal model and detecting the level of cholesterol in the CNS of the animal, wherein an increase in the level of cholesterol in the CNS of the animal following administration of the candidate compound indicates that the compound is a compound that increases cholesterol in the CNS of an animal.
